(19) United States
(12) Patent Application Publication
(10) Pub. No.: US 2015/0322049 A1
(43) Pub. Date: Nov. 12, 2015

(54) LEUKOTRIENE PATHWAY ANTAGONISTS FOR THE TREATMENT OF DEMENTIA, COGNITIVE DEFICITS IN PARKINSON'S DISEASE AND/OR LEARNING AND MEMORY DEFICIENCIES IN PARKINSON'S DISEASE

(71) Applicant: Ludwig AIGNER, (US)

(72) Inventors: Ludwig AIGNER, Salzburg (AT); Julia MARSCHALLINGER, Salzburg (AT); Sébastien COUILLARD-DESPRES, Salzburg (AT)

(21) Appl. No.: 14/652,420

(22) PCT Filed: Dec. 13, 2013
(86) PCT No.: PCT/EP2013/076536

§ 371 Date: Jun. 15, 2015

(30) Foreign Application Priority Data
Dec. 13, 2012 (EP) 12197071.9

(51) Int. Cl. C07D 405/04; C07D 209/24; C07D 215/14

(52) U.S. Cl. CPC C07D 405/04; C07D 215/14; C07D 209/24

(57) ABSTRACT

The invention relates to a pharmaceutical composition comprising a leukotriene antagonist or a leukotriene receptor antagonist for the treatment of a subject suffering from dementia in Parkinson's disease. Further, the invention provides a pharmaceutical composition comprising a leukotriene antagonist or a leukotriene receptor antagonist for retrieval or improvement of learning processes or learning deficits and/or the prevention, retrieval or improvement of memory loss or memory impairment in a subject suffering from dementia in Parkinson's disease. The pharmaceutical composition may comprise montelukast for the treatment of subjects suffering from dementia in Parkinson's disease or for retrieval or improvement of learning processes or learning deficits and/or the prevention, retrieval or improvement of memory loss or memory impairment in a Parkinson's disease subject.

The invention relates to a pharmaceutical composition comprising a leukotriene antagonist or a leukotriene receptor antagonist for the treatment of a subject suffering from dementia in Parkinson's disease. Further, the invention provides a pharmaceutical composition comprising a leukotriene antagonist or a leukotriene receptor antagonist for retrieval or improvement of learning processes or learning deficits and/or the prevention, retrieval or improvement of memory loss or memory impairment in a subject suffering from dementia in Parkinson's disease. The pharmaceutical composition can comprise montelukast for the treatment of subjects suffering from dementia in Parkinson's disease or for retrieval or improvement oflearning processes or learning deficits and/or the prevention, retrieval or improvement of memory loss or memory impairment in a Parkinson's disease subject.

A number of documents including patent applications, manufacturer's manuals and scientific publications are cited herein. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

It is known in the art that leukotriene D4 can induce cognitive impairment through the enhancement of CysLT1R-mediated amyloid-beta generation in 3 month old mice (Tang S (2012) Neuropharmacology, epub. 2012.08.026). This increase in amyloid-beta generation can be inhibited by treatment of the mice with the leukotriene antagonist pranlukast. Pranlukast has therefore to be injected bilaterally through a micropipette 2.5 mm below the dura. Such treatment with pranlukast improves learning impairment and memory loss caused by the leukotriene D4 treatment (Tang S (2012) Neuropharmacology, epub. 2012.08.026). This shows that an administration of pranlukast via a sub-dura injection directly to the brain can revert Alzheimer-like phenotypes of a leukotriene D4 induction model in young mice. It has been suggested that leukotriene antagonists might be used as agents for the treatment or prevention of Alzheimer's disease (EP 0 743 064 A1). This suggestion followed the rational, that chronic inflammation is a major component in Alzheimer's disease. In this line, it has been shown that treatment with anti-inflammatory agents such as indomethacin can stabilize mental functions in Alzheimer's disease patients (Schnabel, 1993, Science, 260: 1719-1720).

It has been suggested that the leukotriene receptor antagonist montelukast potentiates the protective effect of rofecoxib against kainic acid-induced cognitive dysfunction in rats. This was concluded from a study which assessed the use of montelukast in a combination treatment with the nonsteroidal anti-inflammatory drug rofecoxib in young rats (Kumar A (2012) Pharm., Bioch. and Behay., 103: 43-52). The study shows that intraperitoneally administered montelukast can be beneficial in regard to memory functions when administered in a combination treatment with rofecoxib. A mono-therapy with montelukast as active ingredient was not discussed by Kumar and colleagues (2012).

In an in vitro model it was shown that the inhibition of the leukotriene receptor can boost neural progenitor proliferation (Huber et al. (2011) Cell Physiol Biochem, 28:793-804). While an increased proliferation of neural progenitor cells (NPC) was found upon treatment of NPC cultures with montelukast in concentrations of 10 μM and 15 μM a significant reduction in NPC proliferation was found for montelukast concentrations of 50 and 100 μM. Treatment of the NPC cultures with 1 μM or 30 μM montelukast did not alter their proliferation rate. In line with this finding, Huber et al. showed that 15 μM montelukast can significantly increase neural stem cell (NSC) proliferation while 30 μM and 100 μM montelukast lead to a significantly reduced proliferative potential of the NSCs. In the same study, it was reported that a treatment of NPCs with Leukotriene C4 or Leukotriene D4 has no influence on the proliferation of NPCs.

Parkinson's disease (PD) is one of the major neurodegenerative disorders and characterized by progressive neuronal loss, in particular of dopaminergic neurons of the substantia nigra, leading to motor disturbances such as tremor, rigidity, slowness of movement, and postural instability. There is an increasing number of atypical Parkinson syndromes and subclasses of Parkinson's Disease, which are associated with learning and memory deficits. Typical examples are Parkinson's Disease Dementia (PDD) (Goetz et al (2008) Ann Neurol Suppl 2:S81-92), Lewy Body Dementia (LBD) (Meeus et al (2012) Arch Neurol. 69(9):1113-8), and Multi-Systems Atrophy (MSA) (Kao et al. (2009) Alzheimer Dis Assoc Disord. 23(4):365-70). Furthermore, various animal models of PD like diseases exhibit impaired performance in standardized tests of spatial learning and memory skills (Magen et al. (2012) Eur J Neurosci. 35(6):870-82). One mechanism discussed to be involved in cognitive flexibility and learning and memory performances in the adult brain is the rate of hippocampal neurogenesis. While some studies speculate that PD is associated with reduced neurogenesis within the hippocampus (Winner et al. (2004) J Neuropathol Exp Neurol. (11):1155-66; Kohl et al. (2012) Eur J Neurosci. 35(1):10-9), other studies demonstrated the opposite, i.e. induced levels of neurogenesis (Park et al., (2010) Exp Neurol. 222(2):267-76; Winner et al., (2006) Exp Neurol.; 197(1):113-21.) and would therefore hamper pharmacological stimulation of neurogenesis with the aim to promote cognitive function and in particular learning in Parkinson's disease or PD-like disorders.

In summary, the specific alterations in the PD brain and the underlying mechanisms subsequently leading to the observed learning and memory deficits are not yet fully elucidated.

Thus, there is a need for treatment options for dementia as well as specific memory impairments or memory loss in Parkinson's disease. The technical problem underlying the present invention is, accordingly, the provision of a treatment for patients suffering from dementia in Parkinson's disease and the prevention or treatment of memory impairments or memory loss in said patients.

The term “dementia in Parkinson's disease” as employed herein is to be understood to comprise, in particular, Parkinson's Disease Dementia, Lewy Body Dementia, Multi-Systems-Atrophy.

The solution to this technical problem is provided by the embodiments as defined herein and as characterized in the claims.

This invention relates to a pharmaceutical composition comprising a leukotriene antagonist or a leukotriene receptor antagonist for the treatment of a subject suffering from dementia in Parkinson's disease. Further, this invention also relates to a pharmaceutical composition comprising a leukotriene antagonist or a leukotriene receptor antagonists for retrieval or improvement of learning processes or learning deficits and/or the prevention, retrieval or improvement of memory loss or memory impairment in a subject suffering from dementia in Parkinson's disease.

The present invention demonstrates in an animal model of dementia in Parkinson's disease (aged adult rats) that leukotriene pathway antagonists can surprisingly be used in treating dementia in Parkinson's disease, in particular but not limiting in aged, preferably older than 55 years old, Parkinson's disease patients. Aged adult rats suffer from learning deficits and memory impairment. Surprisingly, the leukotriene pathway antagonists of this invention can retrieve or improve said learning deficits and can further prevent, retrieve or improve memory loss or memory impairment in this animal model of dementia in Parkinson's disease. Surprisingly, in Parkinson's disease, cognitive function and in particular learning is promoted in particular by treatment with montelukast. In contrast, the treatment of young adult rats with leukotriene receptor pathway antagonists does not lead to improvements of learning and memory functions in said young adult rats. A strong increase of 5-Lox expression has been reported in the hippocampus of patients suffering of Alzheimer's disease (AD), as well as in AD transgenic mouse models. The hippocampus, which is involved in the cognitive processing, is severely affected by the AD pathological processes. The increase of 5-Lox expression leads to a local increase in leukotriene concentrations. In accordance with the present invention, it could furthermore be shown that 5-Lox expression is also increased in the hippocampus of the Thy1-aSyn mouse (i.e. a non-human Parkinson's model that also shows cognitive deficits from 4 month of age and is, accordingly a model for Parkinson's dementias), although the hippocampus is not a primary target of this disease. Accordingly, it is surprisingly found in accordance with the present invention that leukotriene receptor antagonists, such as montelukast, also constitutes a valid intervention in the alleviation of cognitive impairments of Parkinson's disease/Parkinson's dementia and atypical forms of cognitive disorders/dysfunction in Parkinson's disease/Parkinson's dementia.

Cognitive dysfunction in Parkinsons's disease (PD) is a prominent non-motor symptom in PD, highly contributing to morbidity and mortality in this disease (Forsaa, 2010, Neurology 75(14):1270-1276). The etiologies of cognitive impairments in PD patients are heterogenous and include executive dysfunctions, thought disorders, and very often manifest in dementia, which affects up to 80% of patients (Uc, 2005, Neurology 65:1907-1913; Aarsland, 2011, Neurosci Rep 11:371-378; Narayanan, 2013, Rev Neurosci 24(3):267-278). Yet, these cognitive impairments/dementia and the specific, isolated learning deficits seen in Parkinsons's disease constitute a specific, isolated class of dementia/learning deficits/cognitive impairments that are not to be compared with dementias seen in other neurodegenerative disorders, like Alzheimer's disease. Accordingly, even the structural changes leading to dementia in Parkinsons's disease are not equivalent and clearly differ from the structural brain changes in Alzheimer's disease (AD) or in elderly individuals suffering from age-related dementia. Several studies have shown an association between the number of cortical Lewy bodies and dementia (Hurtig, 2000, Neurology 54(10):1916-1921; Aarsland, 2005, Mov Disord 20:1255-1263). The Braak hypothesis states that Lewy body accumulation spreads from the pons and brain stem via the forebrain and limbic system, to (in the final stage) the neocortex. These stages might progress in parallel with cognitive decline (Braak, 2005, Neurology, 64(8):1404-1410). A small prospective study has demonstrated that staging of pathological changes in Lewy bodies was the strongest correlate of the rate of cognitive decline in Parkinsons's disease patients (Aarsland, 2005, Mov Disord 20:1255-1263). Some studies, though, discuss Alzheimer's-type pathological changes, e.g tau and amyloid deposition in the brain, to contribute to dementia in PD (Kempster, 2010, Brain 133:1755-1762; Irwin, Ann Neurol, 72:587-598). However, it is unlikely that these AD-pathology related features are the ultimate cause for dementia in Parkinsons's disease patients. Patients with pure dementia of Lewy bodies (belonging to the atypical Parkinson syndromes and demonstrating a subclass of Parkinsons's disease) do not exhibit the pathological changes seen in Alzheimer's disease, but still have a global dementia with deficits in memory, attention, language, psychomotor performance, and executive functions, and strikingly severe deficits in visuospatial and visuoconstructive abilities (Salmon, 1996, Brain Cogn 31(2):148-65).

The present invention, therefore and in particular, relates to a treatment/medical intervention of the specific dementia or specific learning disorder/cognitive impairment in Parkinson's disease by leukotriene antagonists or leukotriene receptor antagonists, like and in particular Montelukast. Specific dementia in Parkinson's disease includes but is not limited to Parkinson's Disease Dementia (PDD), Lewy Body Dementia (LBD), Multi-Systems-Atrophy (MSA). The etiologies of cognitive impairments in PD patients are heterogeneous and include executive dysfunctions, thought disorders, and very often manifest in dementia, which affects up to 80% of patients (Uc, 2005, Neurology 65:1907-1913; Aarsland, 2011, Neurosci Rep 11:371-378; reviewed in Narayanan, 2013, Rev Neurosci 24(3):267-278), which highlights the fact that no all Parkinson's disease patients suffer of dementia. Indeed, Parkinson's disease dementia(s) and, e.g., Lewy Body Dementias are individual disease entities separate from isolated Parkinson's disease. For example, the WHO international classifications of diseases (ICD10) classifies “dementia with PD” as F20.3, “Lewy Body Dementia” is classified as (F20.8 and as G31.8).

It was particularly surprisingly found in accordance with the present invention that learning deficits in the aged brain can be ameliorated/improved and thereby treated by leukotriene antagonists or leukotriene receptor antagonists. The experimental results as appended herewith document surprisingly that such an amelioration/improvement/treatment appears to be based on an increased neurogenesis in aged adult individuals but surprisingly not in young adult individuals. Therefore, the present invention relates to the use of leukotriene antagonists/leukotriene receptor antagonists, in particular montelukast, for the treatment of cognitive dysfunction, in particular learning deficits, in elderly individuals, in particular in individuals suffering from or prone to suffer from Parkinson's Disease Dementia, Lewy Body Dementia and/or Multi-Systems-Atrophy. The individual to be treated with a leukotriene antagonist or leukotriene receptor antagonist, in particular with montelukast, is preferably an elderly human individual. Said elderly human individual, in particular an individual suffering from or prone to suffer from Parkinson's Disease Dementia, Lewy Body Dementia and/or Multi-Systems-Atrophy, is preferably at least 55 years, more preferably at least 60 and even more preferably at least 65 years old.

The present invention also provides for means and methods to improve memory in individuals suffering from or prone to suffer from Parkinson's Disease Dementia, Lewy Body Dementia and/or Multi-Systems-Atrophy. The term “memory” subsumes many different processes and requires the function of many different brain areas. Overall, human memory provides declarative recall, e.g., for facts and events accessible to conscious recollection, and non-declarative recall, e.g., procedural memory of skills and operations not stored regarding time and place. Research in recent years has provided information necessary to understand many of the various components of memory and has identified associated brain regions. A newly acquired experience initially is susceptible to various forms of disruption. With time, however, the new experience becomes resistant to disruption. This observation has been interpreted to indicate that a labile, working, short-term memory is consolidated into a more stable, long-term memory. Behavioural research has found that the human mind consolidates memory at certain key time intervals. The initial phase of memory consolidation occurs in the first few minutes after an exposure to a new idea or learning experience. The next phase occurs over a longer period of time, such as during sleep. If a learning experience has on-going meaning to us, the next week or so serves as a further period of memory consolidation. In effect, in this phase, the memory moves from short-term to long-term storage. Moreover, various mechanisms have been proposed to account for the formation of long-term memory. A wide range of observations suggest an evolutionarily conserved molecular mechanism involved with the formation of long-term memory. These include increased release of synaptic transmitter, increased number of synaptic receptors, decreased K_{D} of receptors, synthesis of new memory factors either in the presynaptic or postsynaptic element, sprouting of new synaptic connections, increase of the active area in the presynaptic membrane and many others. Synaptic plasticity, the change in the strength of neuronal connections in the brain, is thought to underlie long-term memory storage. Memory consolidation, the process of storing new information in long-term memory is also believed to play a crucial role in a variety of neurological and mental disorders.

Memory impairment can be assessed by various tests including the Mini Mental State Examination (MMSE) test (Folstein M F (1975) J Psychiatr Res, 2(3):189-98) and the General Practitioner Assessment of Cognition (GPCOG) test (Brodaty H (2004) Int J Geriatr Psychiatry 19:870-74). Accordingly, retrieval or improvement of memory loss or memory impairment can be, for example, assessed by improvements in the scores of the above mentioned testing procedures.

Learning is the acquisition of unknown or the modification of known knowledge. This knowledge can comprise information, behaviors, skills, values or preferences. Tests to assess learning abilities are known to the person skilled in the art.

“Leukotriene pathway antagonists” according to this invention may be understood as leukotriene antagonists or leukotriene receptor antagonists.

The present invention also relates, in particular, to the use of montelukast to enhance learning and memory function in particular in individuals suffering from or prone to suffer from Parkinson's Disease (PD) Dementia, Lewy Body Dementia and/or Multi-Systems-Atrophy by elevating the hippocampal levels of neurogenesis.

The present invention relates to a pharmaceutical composition comprising a leukotriene antagonist or a leukotriene receptor antagonist for the treatment of a subject suffering from dementia in/with Parkinson's disease (Parkinson's Disease Dementia), Lewy Body Dementia, Multi-Systems-Atrophy and the like. Further, the present invention relates to a pharmaceutical composition comprising a leukotriene antagonist or a leukotriene receptor antagonist for retrieval or improvement of learning processes or learning deficits and/or the prevention, retrieval or improvement of memory loss or memory impairment in a subject suffering from dementia in Parkinson's disease, Lewy Body Dementia, Multi-Systems-Atrophy and the like. The invention also relates to a method for the treatment of dementia in Parkinson's disease comprising the step of administering a leukotriene antagonist or a leukotriene receptor antagonist. Further, the invention also relates to a method for retrieval or improvement of learning processes or learning deficits and/or the prevention, retrieval or improvement of memory loss or memory impairment in a subject suffering from dementia in Parkinson's disease, Lewy Body Dementia, Multi-Systems-Atrophy and the like.

The invention also relates to a use of a leukotriene pathway antagonist in the manufacture of a medicament for the treatment of a subject suffering from dementia in Parkinson's disease. Further, it also relates to the use of a leukotriene pathway antagonist in the manufacture of a medicament for retrieval or improvement of learning processes or learning deficits and/or the prevention, retrieval or improvement of memory loss or memory impairment in a subject suffering from dementia in Parkinson's disease, Lewy Body Dementia, Multi-Systems-Atrophy and the like.

The subject suffering from dementia in Parkinson's disease, Lewy Body Dementia, Multi-Systems-Atrophy and the like according to this invention can be, and preferably is, a human person suffering from dementia in Parkinson's disease, Lewy Body Dementia, Multi-Systems-Atrophy and the like. Said human person suffering from dementia in Parkinson's disease is preferably an adult person suffering from dementia in Parkinson's disease. Said adult person suffering from dementia in Parkinson's disease can be at least 20, preferably at least 25, 30 or 35, even more preferably at least 40, 45 or 50, even more preferably at least 55 or 60, and most preferably at least 65 years old.

The pharmaceutical composition of the present invention comprising a leukotriene antagonist or a leukotriene receptor antagonist can comprise the leukotriene antagonist or leukotriene receptor antagonist as the sole active ingredient. The herein disclosed pharmaceutical compositions are particularly useful in the medical intervention/treatment or prophylaxis of Parkinson's Disease Dementia, Lewy Body Dementia and/or Multi-Systems-Atrophy.

The pharmaceutical composition of the present invention comprising a leukotriene antagonist or a leukotriene receptor antagonist can be administered in monotherapy.

The leukotriene antagonist or the leukotriene receptor antagonist to be used in accordance with the present invention is preferably a compound of one the following formulae (I), (II) or (III):

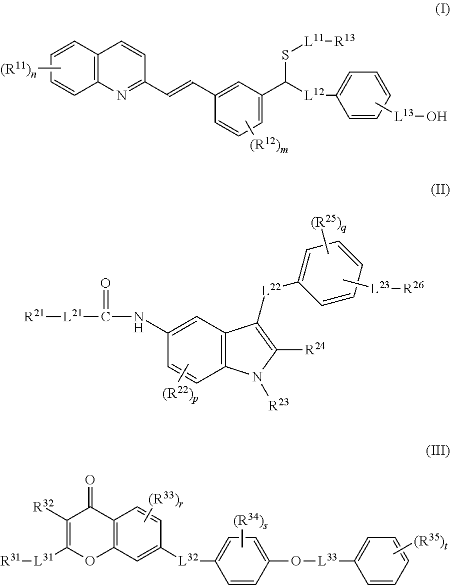

or a pharmaceutically acceptable salt or solvate thereof.

In formula (I), each R¹¹ is independently selected from halogen (e.g., —F, —Cl, —Br, or —I), —CF₃, —CN, —NO₂, —N₃, C₁₋₄ alkyl, —OH, —O(C₁₋₄ alkyl), —SH, —S(C₁₋₄ alkyl), —NH₂, —NH(C₁₋₄ alkyl), or —N(C₁₋₄ alkyl)(C₁₋₄ alkyl). Preferably, each R¹¹ is independently selected from halogen, —CF₃, or —CN. More preferably, each R¹¹ is independently halogen. Most preferably, each R¹¹ is —Cl.

Each R¹² is independently selected from C₁₋₄ alkyl, halogen, —CF₃, —CN, —NO₂, —N₃, —OH, —O(C₁₋₄ alkyl), —SH, —S(C₁₋₄ alkyl), —NH₂, —NH(C₁₋₄ alkyl), or —N(C₁₋₄ alkyl)(C₁₋₄ alkyl). Preferably, each R¹² is independently selected from C₁₋₄ alkyl, halogen, —CF₃, —CN, —OH, —O(C₁₋₄ alkyl), —NH₂, —NH(C₁₋₄ alkyl), or —N(C₁₋₄ alkyl)(C₁₋₄ alkyl).

R¹³ is selected from —COOH, tetrazolyl (particularly tetrazol-5-yl, including 1H-tetrazol-5-yl and 2H-tetrazol-5-yl), —SO₃H, —SO—NH₂, —SO₂—NH₂, —CO—NH—OH, —CO—NH—CN, —CO—NH—SO—(C₁₋₄ alkyl), —CO—NH—SO₂—(C₁₋₄ alkyl), —CO—NH—SO—(C₃₋₇ cycloalkyl), —CO—NH—SO₂—(C₃₋₇ cycloalkyl), —CO—NH—SO-aryl, —CO—NH—SO₂-aryl, —CO—NH—SO-heteroaryl, —CO—NH—SO₂-heteroaryl, —CO—NH—SO—(C₁₋₄ alkylene)aryl, —CO—NH—SO₂—(C₁₋₄ alkylene)aryl, —CO—NH—SO—(C₁₋₄ alkylene)heteroaryl, or —CO—NH—SO₂—(C₁₋₄ alkylene)heteroaryl, wherein the aryl moiety or the heteroaryl moiety comprised in any of the aforementioned groups is optionally substituted with one or more (e.g., one, two, or three) groups independently selected from C₁₋₄ alkyl, halogen, —CF₃, —CN, —NO₂, —N₃, —OH, —O(C₁₋₄ alkyl), —SH, —S(C₁₋₄ alkyl), —NH₂, —NH(C₁₋₄ alkyl), or —N(C₁₋₄ alkyl)(C₁₋₄ alkyl). Preferably, R¹³ is selected from —COOH, tetrazolyl (particularly tetrazol-5-yl, including 1H-tetrazol-5-yl and 2H-tetrazol-5-yl), —SO₃H, —SO—NH₂, —SO₂—NH₂, —CO—NH—OH, or —CO—NH—CN. Most preferably, R¹³ is —COOH.

L¹¹ is C₁₋₆ alkylene or C₂₋₆ alkenylene, wherein said alkylene or said alkenylene is optionally substituted with one or more groups (e.g., one, two, or three groups) independently selected from halogen, —CF₃, —CN, —OH, —O(C₁₋₄ alkyl), —NH₂, —NH(C₁₋₄ alkyl), or —N(C₁₋₄ alkyl)(C₁₋₄ alkyl), and further wherein one —CH₂— unit comprised in said alkylene or said alkenylene is optionally replaced by C₃₋₇ cycloalkylene. Said cycloalkylene is preferably a 1,1-cycloalkylene and more preferably a 1,1-cyclopropylene group. It is preferred that L¹¹ is C₁₋₄ alkylene, wherein one —CH₂— unit comprised in said C₁₋₄ alkylene is replaced by a 1,1-cycloalkylene having 3 to 7 carbon atoms (preferably by 1,1-cyclopropylene), and further wherein said C₁₋₄ alkylene is optionally substituted with one or more groups independently selected from halogen, —CF₃, —CN, —OH, —O(C₁₋₄ alkyl), —NH₂, —NH(C₁₋₄ alkyl), or —N(C₁₋₄ alkyl)(C₁₋₄ alkyl). More preferably, L¹¹ is C₁₋₄ alkylene, wherein one —CH₂— unit comprised in said C₁₋₄ alkylene is replaced by 1,1-cyclopropylene. Most preferably, L¹¹ is —CH₂-(1,1-cyclopropylene)-CH₂— (i.e.,

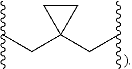 [SMILES: *CC1(C*)CC1]

L¹² L is C₁₋₆ alkylene or C₂₋₆ alkenylene, wherein said alkylene or said alkenylene is optionally substituted with one or more groups (e.g., one, two, or three groups) independently selected from halogen, —CF₃, —CN, —OH, —O(C₁₋₄ alkyl), —NH₂, —NH(C₁₋₄ alkyl), or —N(C₁₋₄ alkyl)(C₁₋₄ alkyl). Preferably, L¹² is C₁₋₄ alkylene (particularly ethylene or n-propylene) optionally substituted with one or more groups independently selected from halogen, —CF₃, —CN, —OH, —O(C₁₋₄ alkyl), —NH₂, —NH(C₁₋₄ alkyl), or —N(C₁₋₄ alkyl)(C₁₋₄ alkyl). More preferably, L¹² is —CH₂CH₂— or —CH₂CH₂CH₂—. Most preferably, L¹² is —CH₂CH₂—.

L¹³ is C₁₋₆ alkylene or C₂₋₆ alkenylene, wherein said alkylene or said alkenylene is optionally substituted with one or more groups (e.g., one, two, or three groups) independently selected from halogen, —CF₃, —CN, —OH, —O(C₁₋₄ alkyl), —NH₂, —NH(C₁₋₄ alkyl), or —N(C₁₋₄ alkyl)(C₁₋₄ alkyl). Preferably, L¹³ is C₁₋₄ alkylene (particularly methylene or ethylene) optionally substituted with one or more groups independently selected from halogen, —CF₃, —CN, —OH, —O(C₁₋₄ alkyl), —NH₂, —NH(C₁₋₄ alkyl), or —N(C₁₋₄ alkyl)(C₁₋₄ alkyl). More preferably, L¹³ is C₁₋₄ alkylene. Most preferably, L¹³ is dimethylmethylene (i.e., —C(—CH₃)₂—).

n is an integer of 0 to 4. Preferably, n is 0, 1 or 2. More preferably, n is 1.

Accordingly, it is particularly preferred that the compound of formula (I) comprises one substituent R¹¹ (i.e., n is 1), wherein R¹¹ is halogen (preferably —Cl), and further wherein said halogen (or said —Cl) is preferably bound to position 7 of the quinoline ring comprised in the compound of formula (I).

m is an integer of 0 to 4. Preferably, m is 0, 1 or 2. More preferably, m is 0 or 1. Most preferably, m is 0.

It is to be understood that, if n is 0, there are no substituents R¹¹ on the quinoline moiety comprised in the compound of formula (I), i.e. the corresponding quinoline ring atoms are substituted with hydrogen. Likewise, if m is 0, there are no substituents R¹² on the corresponding phenyl moiety comprised in the compound of formula (I).

A particularly preferred compound of formula (I) is the following compound or a pharmaceutically acceptable salt or solvate thereof:

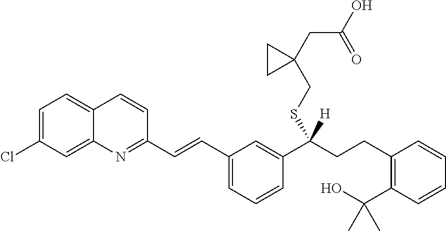 [SMILES: CC(C)(O)c1ccccc1CC[C@@H](SCC1(CC(=O)O)CC1)c1cccc(/C=C/c2ccc3ccc(Cl)cc3n2)c1]

Accordingly, it is particularly preferred that the compound of formula (I) is montelukast or a pharmaceutically acceptable salt or solvate thereof. A preferred pharmaceutically acceptable salt of montelukast is the monosodium salt (i.e., montelukast sodium). Montelukast, as also employed in the appended examples, is particularly preferred in the medical intervention of Parkinson's Disease Dementia, Lewy Body Dementia, Multi-Systems-Atrophy and the like.

The leukotriene antagonist or the leukotriene receptor antagonist may also be a compound of the following formula (II):

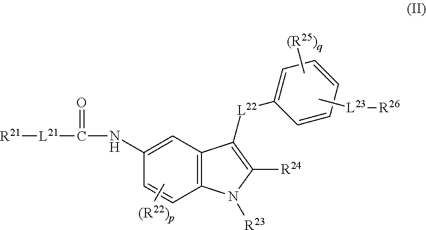

or a pharmaceutically acceptable salt or solvate thereof.

R²¹ is selected from C₁₋₆ alkyl (e.g., butyl or pentyl), C₂₋₆ alkenyl, C₂₋₆ alkynyl, C₃₋₇ cycloalkyl (e.g., cyclopentyl), —(C₁₋₄ alkylene)-(C₃₋₇ cycloalkyl) (e.g., cyclopentylmethyl), phenyl, or —(C₁₋₄ alkylene)-phenyl (e.g., benzyl). Preferably, R²¹ is C₃₋₆ alkyl (e.g., butyl or pentyl), C₃₋₇ cycloalkyl, or —(C₁₋₄ alkylene)-(C₃₋₇ cycloalkyl). More preferably, R²¹ is C₃₋₇ cycloalkyl. Most preferably, R²¹ is cyclopentyl.

Each R²² is independently selected from C₁₋₄ alkyl, halogen, —CF₃, —CN, —NO₂, —N₃, —OH, —O(C₁₋₄ alkyl), —SH, —S(C₁₋₄ alkyl), —NH₂, —NH(C₁₋₄ alkyl), or —N(C₁₋₄ alkyl)(C₁₋₄ alkyl). Preferably, each R²² is independently selected from C₁₋₄ alkyl, halogen, —CF₃, —CN, —OH, —O(C₁₋₄ alkyl), —NH₂, —NH(C₁₋₄ alkyl), or —N(C₁₋₄ alkyl)(C₁₋₄ alkyl).

R²³ is selected from hydrogen, C₁₋₆ alkyl (e.g., methyl, ethyl, n-propyl, or isopropyl), C₂₋₆ alkenyl (e.g., allyl), C₂₋₆ alkynyl (e.g., propargyl), —(C₁₋₄ alkylene)-O—(C₁₋₄ alkyl) (e.g., 2-methoxyethyl), —(C₁₋₄ alkylene)-COOH (e.g., carboxymethyl or carboxyethyl), —(C₁₋₄ alkylene)-CONH₂, —(C₁₋₄ alkylene)-CONH—(C₁₋₄ alkyl) (e.g., —CH₂—CONH—CH₂CH₃), —(C₁₋₄ alkylene)-CON(C₁₋₄ alkyl)(C₁₋₄ alkyl) (e.g., —CH₂—CON(CH₃)—CH₃), C₃₋₇ cycloalkyl (e.g., cyclopropyl or cyclopentyl), —(C₁₋₄ alkylene)-(C₃₋₇ cycloalkyl) (e.g., cyclopropylmethyl or cyclopentylmethyl), —CO—(C₁₋₄ alkyl) (e.g., acetyl), or —(C₁₋₄ alkylene)-phenyl (e.g., benzyl). Preferably, R²³ is C₁₋₄ alkyl. More preferably, R²³ is methyl.

R²⁴ is selected from hydrogen, C₁₋₄ alkyl, halogen, —CF₃, —CN, —NO₂, —N₃, —OH, —O(C₁₋₄ alkyl), —SH, —S(C₁₋₄ alkyl), —NH₂, —NH(C₁₋₄ alkyl), or —N(C₁₋₄ alkyl)(C₁₋₄ alkyl). Preferably, R²⁴ is hydrogen.

Each R²⁵ is independently selected from C₁₋₄ alkyl, halogen, —CF₃, —CN, —NO₂, —N₃, —OH, —O(C₁₋₄ alkyl), —SH, —S(C₁₋₄ alkyl), —NH₂, —NH(C₁₋₄ alkyl), or —N(C₁₋₄ alkyl)(C₁₋₄ alkyl). Preferably, each R²⁵ is independently selected from C₁₋₄ alkyl, halogen, —CF₃, —CN, —OH, —O(C₁₋₄ alkyl), —NH₂, —NH(C₁₋₄ alkyl), or —N(C₁₋₄ alkyl)(C₁₋₄ alkyl).

R²⁶ is selected from —COOH, tetrazolyl (particularly tetrazol-5-yl, including 1H-tetrazol-5-yl and 2H-tetrazol-5-yl), —SO₃H, —SO—NH₂, —SO₂—NH₂, —CO—NH—OH, —CO—NH—CN, —CO—NH—SO—(C₁₋₄ alkyl), —CO—NH—SO₂—(C₁₋₄ alkyl), —CO—NH—SO—(C₃₋₇ cycloalkyl), —CO—NH—SO₂—(C₃₋₇ cycloalkyl), —CO—NH—SO-aryl, —CO—NH—SO₂-aryl, —CO—NH—SO-heteroaryl, —CO—NH—SO₂-heteroaryl, —CO—NH—SO—(C₁₋₄ alkylene)aryl, —CO—NH—SO₂—(C₁₋₄ alkylene)aryl, —CO—NH—SO—(C₁₋₄ alkylene)heteroaryl, or —CO—NH—SO₂—(C₁₋₄ alkylene)heteroaryl, wherein the aryl moiety or the heteroaryl moiety comprised in any of the aforementioned groups is optionally substituted with one or more (e.g., one, two, or three) groups independently selected from C₁₋₄ alkyl, halogen, —CF₃, —CN, —NO₂, —N₃, —OH, —O(C₁₋₄ alkyl), —SH, —S(C₁₋₄ alkyl), —NH₂, —NH(C₁₋₄ alkyl), or —N(C₁₋₄ alkyl)(C₁₋₄ alkyl).

Preferably, R²⁶ is selected from —COOH, tetrazolyl, —CO—NH—SO₂—(C₁₋₄ alkyl), —CO—NH—SO₂—(C₃₋₇ cycloalkyl), —CO—NH—SO₂-aryl, —CO—NH—SO₂-heteroaryl, —CO—NH—SO₂—(C₁₋₄ alkylene)aryl, or —CO—NH—SO₂—(C₁₋₄ alkylene)heteroaryl, wherein the aryl moiety or the heteroaryl moiety comprised in any of the aforementioned groups is optionally substituted with one or more (e.g., one, two, or three) groups independently selected from C₁₋₄ alkyl, halogen, —CF₃, —CN, —NO₂, —N₃, —OH, —O(C₁₋₄ alkyl), —SH, —S(C₁₋₄ alkyl), —NH₂, —NH(C₁₋₄ alkyl), or —N(C₁₋₄ alkyl)(C₁₋₄ alkyl). More preferably, R²⁶ is selected from —COOH, —CO—NH—SO₂-aryl, —CO—NH—SO₂-heteroaryl, —CO—NH—SO₂—(C₁₋₄ alkylene)aryl, or —CO—NH—SO₂—(C₁₋₄ alkylene)heteroaryl, wherein the aryl moiety or the heteroaryl moiety comprised in any of the aforementioned groups is optionally substituted with one or more (e.g., one, two, or three) groups independently selected from C₁₋₄ alkyl, halogen, —CF₃, —CN, —NO₂, —N₃, —OH, —O(C₁₋₄ alkyl), —SH, —S(C₁₋₄ alkyl), —NH₂, —NH(C₁₋₄ alkyl), or —N(C₁₋₄ alkyl)(C₁₋₄ alkyl). Even more preferably, R²⁶ is —CO—NH—SO₂-phenyl, wherein the phenyl moiety comprised in said —CO—NH—SO₂-phenyl is optionally substituted with one or two (preferably one) groups independently selected from C₁₋₄ alkyl, halogen, —CF₃, —CN, —NO₂, —N₃, —OH, —O(C₁₋₄ alkyl), —SH, —S(C₁₋₄ alkyl), —NH₂, —NH(C₁₋₄ alkyl), or —N(C₁₋₄ alkyl)(C₁₋₄ alkyl). A particularly preferred example of R²⁶ is —CO—NH—SO₂-(o-methylphenyl).

L²¹ is a covalent bond, —O—, —S—, —NH—, or —N(C₁₋₄ alkyl)-. Preferably, L²¹ is a covalent bond, —O—, —NH—, or —N(C₁₋₄ alkyl)-. More preferably, L²¹ is —O—.

L²² is a covalent bond or C₁₋₄ alkylene. Preferably, L²² is a covalent bond, methylene, or ethylene. Most preferably, L²² is methylene.

L²³ is a covalent bond or C₁₋₄ alkylene. Preferably, L²³ is a covalent bond or methylene. Most preferably, L²³ is a covalent bond.

It is to be understood that, if L²³ is a covalent bond, the moiety —R²⁶ is directly bound to the phenyl moiety comprised in the compound of formula (II).

It is furthermore preferred that the moiety -L²³-R²⁶ is bound to the phenyl moiety comprised in the compound of formula (II) in meta or in para position with respect to the group L²². Most preferably, the moiety -L²³-R²⁶ is bound to the phenyl moiety comprised in the compound of formula (II) in para position with respect to L²².

p is an integer of 0 to 3. Preferably, p is 0, 1 or 2. More preferably, p is 0 or 1. Most preferably, p is 0.

q is an integer of 0 to 4. Preferably, q is 0, 1 or 2. More preferably, q is 0 or 1. Most preferably, q is 0.

It is to be understood that, if p is 0, there are no substituents R²² on the indole moiety comprised in the compound of formula (II), i.e. the corresponding indole ring atoms are substituted with hydrogen. Likewise, if q is 0, there are no substituents R²⁵ on the corresponding phenyl moiety comprised in the compound of formula (II).

A particularly preferred compound of formula (II) is the following compound or a pharmaceutically acceptable salt or solvate thereof:

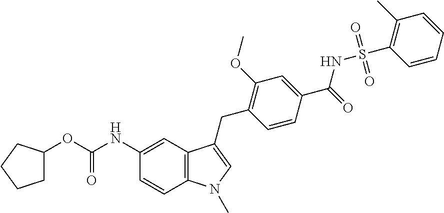 [SMILES: COc1cc(C(=O)NS(=O)(=O)c2ccccc2C)ccc1Cc1cn(C)c2ccc(NC(=O)OC3CCCC3)cc12]

Accordingly, it is particularly preferred that the compound of formula (II) is zafirlukast or a pharmaceutically acceptable salt or solvate thereof.

The leukotriene antagonist or the leukotriene receptor antagonist may also be a compound of the following formula (III):

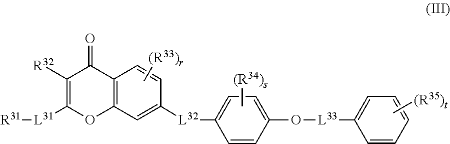

or a pharmaceutically acceptable salt or solvate thereof.

R³¹ is selected from —COOH, tetrazolyl (particularly tetrazol-5-yl, including 1H-tetrazol-5-yl and 2H-tetrazol-5-yl), —SO₃H, —SO—NH₂, —SO₂—NH₂, —CO—NH—OH, —CO—NH—CN, —CO—NH—SO—(C₁₋₄ alkyl), —CO—NH—SO₂—(C₁₋₄ alkyl), —CO—NH—SO—(C₃₋₇ cycloalkyl), —CO—NH—SO₂—(C₃₋₇ cycloalkyl), —CO—NH—SO-aryl, —CO—NH—SO₂-aryl, —CO—NH—SO-heteroaryl, —CO—NH—SO₂-heteroaryl, —CO—NH—SO—(C₁₋₄ alkylene)aryl, —CO—NH—SO₂—(C₁₋₄ alkylene)aryl, —CO—NH—SO—(C₁₋₄ alkylene)heteroaryl, or —CO—NH—SO₂—(C₁₋₄ alkylene)heteroaryl, wherein the aryl moiety or the heteroaryl moiety comprised in any of the aforementioned groups is optionally substituted with one or more (e.g., one, two, or three) groups independently selected from C₁₋₄ alkyl, halogen, —CF₃, —CN, —NO₂, —N₃, —OH, —O(C₁₋₄ alkyl), —SH, —S(C₁₋₄ alkyl), —NH₂, —NH(C₁₋₄ alkyl), or —N(C₁₋₄ alkyl)(C₁₋₄ alkyl). Preferably, R³¹ is selected from —COOH, tetrazolyl, —SO₃H, —SO—NH₂, —SO₂—NH₂, —CO—NH—OH, or —CO—NH—CN. More preferably, R³¹ is —COOH or tetrazolyl. Most preferably, R³¹ is tetrazolyl (particularly tetrazol-5-yl, including 1H-tetrazol-5-yl and 2H-tetrazol-5-yl).

R³² is selected from hydrogen, C₁₋₄ alkyl, halogen, —CF₃, —CN, —NO₂, —N₃, —OH, —O(C₁₋₄ alkyl), —SH, —S(C₁₋₄ alkyl), —NH₂, —NH(C₁₋₄ alkyl), or —N(C₁₋₄ alkyl)(C₁₋₄ alkyl). Preferably, R³² is hydrogen.

Each R³³ is independently selected from C₁₋₄ alkyl, halogen, —CF₃, —CN, —NO₂, —N₃, —OH, —O(C₁₋₄ alkyl), —SH, —S(C₁₋₄ alkyl), —NH₂, —NH(C₁₋₄ alkyl), or —N(C₁₋₄ alkyl)(C₁₋₄ alkyl). Preferably, each R³³ is independently selected from C₁₋₄ alkyl, halogen, —CF₃, —CN, —OH, —O(C₁₋₄ alkyl), —NH₂, —NH(C₁₋₄ alkyl), or —N(C₁₋₄ alkyl)(C₁₋₄ alkyl).

Each R³⁴ is independently selected from C₁₋₄ alkyl, halogen, —CF₃, —CN, —NO₂, —N₃, —OH, —O(C₁₋₄ alkyl), —SH, —S(C₁₋₄ alkyl), —NH₂, —NH(C₁₋₄ alkyl), or —N(C₁₋₄ alkyl)(C₁₋₄ alkyl). Preferably, each R³⁴ is independently selected from C₁₋₄ alkyl, halogen, —CF₃, —CN, —OH, —O(C₁₋₄ alkyl), —NH₂, —NH(C₁₋₄ alkyl), or —N(C₁₋₄ alkyl)(C₁₋₄ alkyl).

Each R³⁵ is independently selected from C₁₋₄ alkyl, halogen, —CF₃, —CN, —NO₂, —N₃, —OH, —O(C₁₋₄ alkyl), —SH, —S(C₁₋₄ alkyl), —NH₂, —NH(C₁₋₄ alkyl), or —N(C₁₋₄ alkyl)(C₁₋₄ alkyl). Preferably, each R³⁵ is independently selected from C₁₋₄ alkyl, halogen, —CF₃, —CN, —OH, —O(C₁₋₄ alkyl), —NH₂, —NH(C₁₋₄ alkyl), or —N(C₁₋₄ alkyl)(C₁₋₄ alkyl).

L³¹ is a covalent bond or C₁₋₄ alkylene. Preferably, L³¹ is a covalent bond or methylene. Most preferably, L³¹ is a covalent bond.

L³² is C₁₋₄ alkylene, C₂₋₄ alkenylene, —O—, —S—, —NH—, —N(C₁₋₄ alkyl)-, —CO—, —CO—NH—, —NH—CO—, —CO—N(C₁₋₄ alkyl)-, or —N(C₁₋₄ alkyl)-CO—, wherein one or two —CH₂— units (preferably one —CH₂— unit) comprised in said C₁₋₄ alkylene or said C₂₋₄ alkenylene are each optionally replaced by a group selected independently from —O—, —S—, —NH—, —N(C₁₋₄ alkyl)-, or —CO—. Preferably, L³² is —CO—NH—, —NH—CO—, —CO—N(C₁₋₄ alkyl)-, or —N(C₁₋₄ alkyl)-CO—. More preferably, L³² is —NH—CO— (wherein the —NH— group comprised in said —NH—CO— is bound to the benzopyranone moiety in the compound of formula (III), and the —CO— group comprised in said —NH—CO— is bound to the phenyl moiety in the compound of formula (III)).

L³³ is C₁₋₁₀ alkylene. Preferably, L³³ is C₃₋₈ alkylene. More preferably, L³³ is —(CH₂)₃₋₆. Most preferably, L³³ is —(CH₂)₄—.

r is an integer of 0 to 3. Preferably, r is 0, 1 or 2. More preferably, r is 0 or 1. Most preferably, r is 0.

s is an integer of 0 to 4. Preferably, s is 0, 1 or 2. More preferably, s is 0 or 1. Most preferably, s is 0.

t is an integer of 0 to 5. Preferably, t is 0, 1 or 2. More preferably, t is 0 or 1. Most preferably, t is 0.

It is to be understood that, if r is 0, there are no substituents R³³ on the corresponding fused phenyl moiety comprised in the compound of formula (III), i.e. the corresponding ring atoms are substituted with hydrogen. Likewise, if s or t is 0, there are no substituents R³⁴ or R³⁵ on the corresponding phenyl moieties comprised in the compound of formula (III).

A particularly preferred compound of formula (III) is the following compound or a pharmaceutically acceptable salt or solvate thereof:

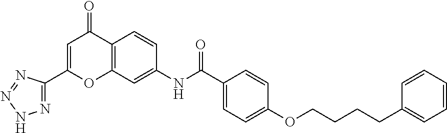 [SMILES: O=C(Nc1ccc2c(=O)cc(-c3nn[nH]n3)oc2c1)c1ccc(OCCCCc2ccccc2)cc1]

Accordingly, it is particularly preferred that the compound of formula (III) is pranlukast or a pharmaceutically acceptable salt or solvate thereof.

The leukotriene antagonist or the leukotriene receptor antagonist to be used in accordance with the present invention is not limited to the compounds of formulae (I), (II) and (III) described herein but also includes further leukotriene antagonists and leukotriene receptor antagonists. Exemplary further leukotriene antagonists include, e.g., zileuton, L 663536 (or MK 886), or the leukotriene antagonists disclosed in EP-A-0743064 (including in particular the compounds of formulae I, II, III, and IV as described and defined in EP-A-0743064, as well as all of the individual compounds described in that document).

As used herein, the term “alkyl” refers to a monovalent saturated aliphatic (i.e., non-aromatic) acyclic hydrocarbon group (i.e., a group consisting of carbon atoms and hydrogen atoms) which may be linear or branched and does not comprise any carbon-to-carbon double bond or any carbon-to-carbon triple bond. A “C₁₋₄ alkyl” denotes an alkyl group having 1 to 4 carbon atoms.

As used herein, the term “alkenyl” refers to a monovalent unsaturated aliphatic acyclic hydrocarbon group which may be linear or branched and comprises at least one carbon-to-carbon double bond while it does not comprise any carbon-to-carbon triple bond.

As used herein, the term “alkynyl” refers to a monovalent unsaturated aliphatic acyclic hydrocarbon group which may be linear or branched and comprises at least one carbon-to-carbon triple bond and optionally one or more carbon-to-carbon double bonds.

As used herein, the term “alkylene” refers to a divalent saturated aliphatic acyclic hydrocarbon group which may be linear or branched and does not comprise any carbon-to-carbon double bond or any carbon-to-carbon triple bond.

As used herein, the term “alkenylene” refers to a divalent unsaturated aliphatic acyclic hydrocarbon group which may be linear or branched and comprises at least one carbon-to-carbon double bond while it does not comprise any carbon-to-carbon triple bond.

As used herein, the term “cycloalkyl” refers to a monovalent cyclic saturated aliphatic hydrocarbon group which does not comprise any carbon-to-carbon double bond or any carbon-to-carbon triple bond. Non-limiting examples of cycloalkyl groups are cyclopropyl, cyclobutyl, cyclopentyl, cyclohexyl or cycloheptyl.

As used herein, the term “cycloalkylene” refers to a divalent cyclic saturated aliphatic hydrocarbon group which does not comprise any carbon-to-carbon double bond or any carbon-to-carbon triple bond. Non-limiting examples of cycloalkylene groups are cyclopropylene, cyclobutylene, cyclopentylene, cyclohexylene or cycloheptylene. The cycloalkylene groups may also be 1,1-cycloalkylene groups, such as, e.g., 1,1-cyclopropylene

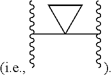 [SMILES: *C1(*)CC1]

As used herein, the term “aryl” refers to a monovalent aromatic hydrocarbon group, including monocyclic as well as bridged ring and/or fused ring systems, containing at least one aromatic ring. “Aryl” may, for example, refer to phenyl, naphthyl or anthracenyl.

As used herein, the term “heteroaryl” refers to a monovalent aromatic ring group, including monocyclic as well as bridged ring and/or fused ring systems, containing at least one aromatic ring which comprises one or more (such as, e.g., one, two, or three) ring heteroatoms independently selected from O, S, or N. The “heteroaryl” may, e.g., have 5 to 14 ring atoms, particularly 5 or 6 ring atoms. “Heteroaryl” may, for example, refer to thiophenyl (thienyl), furanyl (furyl), pyrrolyl, imidazolyl, pyrazolyl, pyridinyl (pyridyl; including, e.g., 2-pyridyl, 3-pyridyl, and 4-pyridyl), pyrazinyl, pyrimidinyl, pyridazinyl, oxazolyl, isoxazolyl, or furazanyl.

As used herein, the term “halogen” refers to —F, —CI, —Br or —I.

For a person skilled in the field of synthetic chemistry, various ways for the preparation of the leukotriene antagonists or the leukotriene receptor antagonists of the present invention, including in particular the compounds of formula (I), (II) or (III), will be readily apparent. The compounds of formula (I), (II) or (III) can, for example, be prepared in accordance with or in analogy to the methods described in EP-A-0480717, U.S. Pat. No. 4,859,692 and U.S. Pat. No. 4,994,479.

The scope of the present invention embraces all pharmaceutically acceptable salt forms of the leukotriene antagonists or the leukotriene receptor antagonists of the present invention, including the compounds of formula (I), (II) or (III), which may be formed, e.g., by protonation of an atom carrying an electron lone pair which is susceptible to protonation, such as an amino group, with an inorganic or organic acid, or as a salt of a carboxylic acid group with a physiologically acceptable cation as they are well-known in the art. Exemplary base addition salts comprise, for example: alkali metal salts such as sodium or potassium salts; alkaline earth metal salts such as calcium or magnesium salts; ammonium salts; aliphatic amine salts such as trimethylamine, triethylamine, dicyclohexylamine, ethanolamine, diethanolamine, triethanolamine, procaine salts, meglumine salts, diethanol amine salts or ethylenediamine salts; aralkyl amine salts such as N,N-dibenzylethylenediamine salts, benetamine salts; heterocyclic aromatic amine salts such as pyridine salts, picoline salts, quinoline salts or isoquinoline salts; quaternary ammonium salts such as tetramethylammonium salts, tetraethylammonium salts, benzyltrimethylammonium salts, benzyltriethylammonium salts, benzyltributylammonium salts, methyltrioctylammonium salts or tetrabutylammonium salts; and basic amino acid salts such as arginine salts or lysine salts. Exemplary acid addition salts comprise, for example, mineral acid salts such as hydrochloride, hydrobromide, hydroiodide, sulfate salts, nitrate salts, phosphate salts (such as, e.g., phosphate, hydrogenphosphate, or dihydrogenphosphate salts), carbonate salts, hydrogencarbonate salts or perchlorate salts; organic acid salts such as acetate, propionate, butyrate, pentanoate, hexanoate, heptanoate, octanoate, cyclopentanepropionate, undecanoate, lactate, maleate, oxalate, fumarate, tartrate, malate, citrate, nicotinate, benzoate, salicylate or ascorbate salts; sulfonate salts such as methanesulfonate, ethanesulfonate, 2-hydroxyethanesulfonate, benzenesulfonate, p-toluenesulfonate (tosylate), 2-naphthalenesulfonate, 3-phenylsulfonate, or camphorsulfonate salts; and acidic amino acid salts such as aspartate or glutamate salts.

Moreover, the scope of the invention embraces the leukotriene antagonists or the leukotriene receptor antagonists of the present invention, including the compounds of formula (I), (II) or (III), in any solvated form, including, e.g., solvates with water, for example hydrates, or with organic solvents such as, e.g., methanol, ethanol or acetonitrile, i.e., as a methanolate, ethanolate or acetonitrilate, respectively, or in the form of any polymorph.

Furthermore, the formulae in the present specification are intended to cover all possible stereoisomers, including enantiomers and diastereomers, of the indicated compounds.

Thus, all stereoisomers of the compounds of the present invention are contemplated as part of the present invention, either in admixture or in pure or substantially pure form. The scope of the leukotriene antagonists or the leukotriene receptor antagonists of the present invention, including the compounds of formula (I), (II) or (III), embraces all of the possible stereoisomers and their mixtures. It particularly embraces the racemic forms and the isolated optical isomers. The racemic forms can be resolved by physical methods, such as, e.g., fractional crystallization, separation or crystallization of diastereomeric derivatives or separation by chiral column chromatography. The individual optical isomers can be obtained from the racemates using conventional methods, such as, e.g., salt formation with an optically active acid followed by crystallization.

The leukotriene antagonists or the leukotriene receptor antagonists described herein may be administered as compounds per se or may be formulated as medicaments. The medicaments/pharmaceutical compositions may optionally comprise one or more pharmaceutically acceptable excipients, such as carriers, diluents, fillers, disintegrants, lubricating agents, binders, colorants, pigments, stabilizers, preservatives, antioxidants, or solubility enhancers.

In particular, the pharmaceutical compositions may comprise one or more solubility enhancers, such as, e.g., poly(ethylene glycol), including poly(ethylene glycol) having a molecular weight in the range of about 200 to about 5,000 Da, ethylene glycol, propylene glycol, non-ionic surfactants, tyloxapol, polysorbate 80, macrogol-15-hydroxystearate, phospholipids, lecithin, dimyristoyl phosphatidylcholine, dipalmitoyl phosphatidylcholine, distearoyl phosphatidylcholine, cyclodextrins, hydroxyethyl-β-cyclodextrin, hydroxypropyl-β-cyclodextrin, hydroxyethyl-γ-cyclodextrin, hydroxypropyl-γ-cyclodextrin, dihydroxypropyl-β-cyclodextrin, glucosyl-α-cyclodextrin, glucosyl-β-cyclodextrin, diglucosyl-β-cyclodextrin, maltosyl-α-cyclodextrin, maltosyl-β-cyclodextrin, maltosyl-γ-cyclodextrin, maltotriosyl-β-cyclodextrin, maltotriosyl-γ-cyclodextrin, dimaltosyl-β-cyclodextrin, methyl-β-cyclodextrin, carboxyalkyl thioethers, hydroxypropyl methylcellulose, hydroxypropylcellulose, polyvinylpyrrolidone, vinyl acetate copolymers, vinyl pyrrolidone, sodium lauryl sulfate, dioctyl sodium sulfosuccinate, or any combination thereof.

The pharmaceutical compositions can be formulated by techniques known to the person skilled in the art, such as the techniques published in Remington's Pharmaceutical Sciences, 20^{th} Edition. The pharmaceutical compositions can be formulated as dosage forms for oral, parenteral, such as intramuscular, intravenous, subcutaneous, intradermal, intraarterial, intracardial, rectal, nasal, topical, aerosol or vaginal administration. Dosage forms for oral administration include coated and uncoated tablets, soft gelatin capsules, hard gelatin capsules, lozenges, troches, solutions, emulsions, suspensions, syrups, elixirs, powders and granules for reconstitution, dispersible powders and granules, medicated gums, chewing tablets and effervescent tablets. Dosage forms for parenteral administration include solutions, emulsions, suspensions, dispersions and powders and granules for reconstitution. Emulsions are a preferred dosage form for parenteral administration. Dosage forms for rectal and vaginal administration include suppositories and ovula. Dosage forms for nasal administration can be administered via inhalation and insufflation, for example by a metered inhaler. Dosage forms for topical administration include creams, gels, ointments, salves, patches and transdermal delivery systems.

The leukotriene antagonists or the leukotriene receptor antagonists of the present invention, including the compounds of formula (I), (II) or (III), or the above described pharmaceutical compositions comprising one ore more leukotriene antagonists or the leukotriene receptor antagonists according to the invention, may be administered to a subject by any convenient route of administration, whether systemically/peripherally or at the site of desired action, including but not limited to one or more of: oral (e.g., as a tablet, capsule, or as an ingestible solution), topical (e.g., transdermal, intranasal, ocular, buccal, and sublingual), parenteral (e.g., using injection techniques or infusion techniques, and including, for example, by injection, e.g., subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, or intrasternal by, e.g., implant of a depot, for example, subcutaneously or intramuscularly), pulmonary (e.g., by inhalation or insufflation therapy using, e.g., an aerosol, e.g., through mouth or nose), gastrointestinal, intrauterine, intraocular, subcutaneous, ophthalmic (including intravitreal or intracameral), rectal, and vaginal.

It is particularly preferred that the leukotriene antagonists or the leukotriene receptor antagonists or pharmaceutical compositions of the present invention are administered orally, e.g., in the form of tablets, capsules, ovules, elixirs, solutions or suspensions, which may further contain flavoring or coloring agents, for immediate-release, delayed-release, modified-release, sustained-release, pulsed-release or controlled-release applications.

The tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycolate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia. Additionally, lubricating agents such as magnesium stearate, stearic acid, glyceryl behenate and talc may be included. Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the agent may be combined with various sweetening or flavoring agents, coloring matter or dyes, with emulsifying and/or suspending agents and with diluents such as water, ethanol, propylene glycol and glycerin, and combinations thereof.

Alternatively, it may also be possible to administer said leukotriene antagonists or leukotriene receptor antagonists or pharmaceutical compositions in the form of a suppository or pessary, or it may be applied topically in the form of a gel, hydrogel, lotion, solution, cream, ointment or dusting powder. The compounds of the present invention may also be dermally or transdermally administered, for example, by the use of a skin patch.

If said leukotriene antagonists or leukotriene receptor antagonists or pharmaceutical compositions are administered parenterally, then examples of such administration include one or more of: intravenously, intraarterially, intraperitoneally, intrathecally, intraventricularly, intraurethrally, intrasternally, intracardially, intracranially, intramuscularly or subcutaneously administering the compounds pharmaceutical compositions, and/or by using infusion techniques. For parenteral administration, the compounds are best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well known to those skilled in the art.

The leukotriene antagonists or the leukotriene receptor antagonists or pharmaceutical compositions may also be administered by the pulmonary route, rectal routes, or the ocular route. For ophthalmic use, they can be formulated as micronized suspensions in isotonic, pH adjusted, sterile saline, or, preferably, as solutions in isotonic, pH adjusted, sterile saline, optionally in combination with a preservative such as a benzylalkonium chloride. Alternatively, they may be formulated in an ointment such as petrolatum.

For topical application to the skin, the leukotriene antagonists or the leukotriene receptor antagonists or pharmaceutical compositions can be formulated as a suitable ointment containing the active compound suspended or dissolved in, for example, a mixture with one or more of the following: mineral oil, liquid petrolatum, white petrolatum, propylene glycol, emulsifying wax and water. Alternatively, they can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, 2-octyldodecanol, benzyl alcohol and water.

Typically, a physician will determine the actual dosage which will be most suitable for an individual subject. The specific dose level and frequency of dosage for any particular individual subject may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the individual subject undergoing therapy.

A proposed, yet non-limiting dose of the leukotriene antagonists or the leukotriene receptor antagonists of the present invention, including in particular the compounds of formula (I), (II) or (III), for oral administration to a human (of approximately 70 kg body weight) may be 0.1 μg to 10 g, preferably 0.1 mg to 1 g, of the active ingredient per unit dose. The unit dose may be administered, for example, 1 to 3 times per day. The unit dose may also be administered 1 to 7 times per week, e.g., with not more than one administration per day. Also for the preferred leukotriene pathway antagonist montelukast to be employed in accordance with this invention, i.e. in the medical intervention of dementios in Parkinson's diease and/or cognitive disorders in Parkinson's disease, a non-limiting dose/treatment scheme of the for oral administration to a human (of approximately 70 kg body weight) may comprise 0.1 μg to 2 g, preferably 0.1 mg to 1 g, more preferably 100 mg to 200 mg, even more preferably 200 mg to 500 mg, of montelukast per unit dose. The unit dose may be administered, for example, 1 to 3 times per day. The unit dose may also be administered 1 to 7 times per week, e.g., with not more than one administration per day. The daily dose is preferably 100 mg to 1000 mg, more preferably 200 to 500 mg. It will be appreciated that it may be necessary to make routine variations to the dosage depending on the age and weight of the patient/subject as well as the severity of the condition to be treated. The precise dose and also the route of administration will ultimately be at the discretion of the attendant physician or veterinarian.

The leukotriene antagonists or the leukotriene receptor antagonists of the present invention, including the compounds of formula (I), (II) or (III), may be administered in the context of a monotherapy or in combination with one or more other pharmaceutically active agents. When a leukotriene antagonist or a leukotriene receptor antagonist of the invention is used in combination with a second pharmaceutically active agent which is active against the same condition or disorder, the dose of each compound may differ from that when the compound is used alone. The combination of a leukotriene antagonist or a leukotriene receptor antagonist of the present invention with one or more other pharmaceutically active agents may comprise the simultaneous/concomitant administration of the pharmaceutically active agents with the leukotriene antagonist or the leukotriene receptor antagonist of the invention. However, sequential/separate administration is also envisaged. It is preferred that the leukotriene antagonist or leukotriene receptor antagonist according to the invention, including particularly the compounds of formula (I), (II) or (III), are to be administered in a monotherapy.

The subject or patient, such as the subject in need of treatment or prevention, may be an animal (e.g., a non-human animal), a vertebrate animal, a mammal, a rodent (e.g., a guinea pig, a hamster, a rat, a mouse), a murine (e.g., a mouse), a canine (e.g., a dog), a feline (e.g., a cat), an equine (e.g., a horse), a primate, a simian (e.g., a monkey or ape), a monkey (e.g., a marmoset, a baboon), an ape (e.g., a gorilla, chimpanzee, orang-utan, gibbon), or a human. The meaning of the terms “eukaryote”, “animal”, “mammal”, etc. is well known in the art and can, for example, be deduced from Wehner and Gehring (1995; Thieme Verlag). In the context of this invention, it is also envisaged that animals are to be treated which are economically, agronomically or scientifically important. Scientifically important organisms include, but are not limited to, mice, rats, and rabbits. Non-limiting examples of agronomically important animals are sheep, cattle and pigs, while, for example, cats and dogs may be considered as economically important animals. Preferably, the subject/patient is a mammal; more preferably, the subject/patient is a human or a non-human mammal (such as, e.g., a guinea pig, a hamster, a rat, a mouse, a rabbit, a dog, a cat, a horse, a monkey, an ape, a marmoset, a baboon, a gorilla, a chimpanzee, an orang-utan, a gibbon, a sheep, cattle, or a pig); most preferably, the subject/patient is a human.

The term “treatment of a disorder or disease” as used herein is well known in the art. “Treatment of a disorder or disease” implies that a disorder or disease is suspected or has been diagnosed in a patient/subject. A patient/subject suspected of suffering from a disorder or disease typically shows specific clinical and/or pathological symptoms which a skilled person can easily attribute to a specific pathological condition (i.e., diagnose a disorder or disease).

“Treatment of a disorder or disease” may, for example, lead to a halt in the progression of the disorder or disease (e.g., no deterioration of symptoms) or a delay in the progression of the disorder or disease (in case the halt in progression is of a transient nature only). “Treatment of a disorder or disease” may also lead to a partial response (e.g., amelioration of symptoms) or complete response (e.g., disappearance of symptoms) of the subject/patient suffering from the disorder or disease. “Amelioration” of a disorder or disease may, for example, lead to a halt in the progression of the disorder or disease or a delay in the progression of the disorder or disease. Such a partial or complete response may be followed by a relapse. It is to be understood that a subject/patient may experience a broad range of responses to a treatment (e.g., the exemplary responses as described herein above).

Treatment of a disorder or disease may, inter alia, comprise curative treatment (preferably leading to a complete response and eventually to healing of the disorder or disease) and palliative treatment (including symptomatic relief).

Also the term “prevention of a disorder or disease” as used herein is well known in the art. For example, a patient/subject suspected of being prone to suffer from a disorder or disease as defined herein may, in particular, benefit from a prevention of the disorder or disease. The subject/patient may have a susceptibility or predisposition for a disorder or disease, including but not limited to hereditary predisposition. Such a predisposition can be determined by standard assays, using, for example, genetic markers or phenotypic indicators. It is to be understood that a disorder or disease to be prevented in accordance with the present invention has not been diagnosed or cannot be diagnosed in the patient/subject (for example, the patient/subject does not show any clinical or pathological symptoms). Thus, the term “prevention” comprises the use of compounds of the present invention before any clinical and/or pathological symptoms are diagnosed or determined or can be diagnosed or determined by the attending physician.

While the invention has been illustrated and described in detail in the drawings and foregoing description, such illustration and description are to be considered illustrative or exemplary and not restrictive. It will be understood that changes and modifications may be made by those of ordinary skill within the scope and spirit of the following claims. In particular, the present invention covers further embodiments with any combination of features from different embodiments described above and below.

The invention also covers all further features shown in the figures individually although they may not have been described in the afore or following description. Also, single alternatives of the embodiments described in the figures and the description and single alternatives of features thereof can be disclaimed from the subject matter of the other aspect of the invention.

Furthermore, in the claims the word “comprising” does not exclude other elements or steps, and the indefinite article “a” or “an” does not exclude a plurality. A single unit may fulfill the functions of several features recited in the claims. The terms “essentially”, “about”, “approximately” and the like in connection with an attribute or a value particularly also define exactly the attribute or exactly the value, respectively. Any reference signs in the claims should not be construed as limiting the scope.

In a particular preferred embodiment, aged Parkinson's disease patients are to be treated with leukotriene antagonists or leukotriene receptor antagonists; in particular Parkinson's disease patients older than 55 years of age are to be treated in particular with montelukast.

In accordance with the above, the appended examples, the appended figures as well as the claims, the present invention in particular and in most preferred embodiments relates to the following items:
1. A pharmaceutical composition comprising a leukotriene antagonist or a leukotriene receptor antagonist for use in the treatment of a subject suffering from dementia in Parkinson's disease.
2. A pharmaceutical composition comprising a leukotriene antagonist or a leukotriene receptor antagonist for use in retrieval or improvement of learning processes or learning deficits and/or the prevention, retrieval or improvement of memory loss or memory impairment in a subject suffering from Parkinson's disease.
3. The pharmaceutical composition of item 2, wherein the subject is a subject suffering from dementia in Parkinson's disease.
4. The pharmaceutical composition of any of items 1 to 3, wherein the subject is a human person.
5. The pharmaceutical composition of any of items 1 to 4, wherein the pharmaceutical composition is administered as a monotherapy.
6. The pharmaceutical composition of any of items 1 to 5, wherein the leukotriene antagonist or leukotriene receptor antagonist is a compound of the following formula:

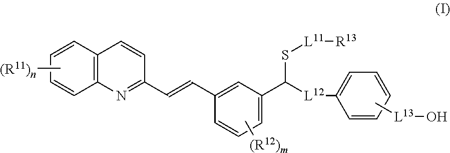

wherein:
each R¹¹ is independently selected from halogen, —CF₃, —CN, —NO₂, —N₃, C₁₋₄ alkyl, —OH, —O(C₁₋₄ alkyl), —SH, —S(C₁₋₄ alkyl), —NH₂, —NH(C₁₋₄ alkyl), or —N(C₁₋₄ alkyl)(C₁₋₄ alkyl);
each R¹² is independently selected from C₁₋₄ alkyl, halogen, —CF₃, —CN, —NO₂, —N₃, —OH, —O(C₁₋₄ alkyl), —SH, —S(C₁₋₄ alkyl), —NH₂, —NH(C₁₋₄ alkyl), or —N(C₁₋₄ alkyl)(C₁₋₄ alkyl);
R¹³ is selected from —COOH, tetrazolyl, —SO₃H, —SO—NH₂, —SO₂—NH₂, —CO—NH—OH, —CO—NH—CN, —CO—NH—SO—(C₁₋₄ alkyl), —CO—NH—SO₂—(C₁₋₄ alkyl), —CO—NH—SO—(C₃₋₇ cycloalkyl), —CO—NH—SO₂—(C₃₋₇ cycloalkyl), —CO—NH—SO-aryl, —CO—NH—SO₂-aryl, —CO—NH—SO-heteroaryl, —CO—NH—SO₂-heteroaryl, —CO—NH—SO—(C₁₋₄ alkylene)aryl, —CO—NH—SO₂—(C₁₋₄ alkylene)aryl, —CO—NH—SO—(C₁₋₄ alkylene)heteroaryl, or —CO—NH—SO₂—(C₁₋₄ alkylene)heteroaryl, wherein the aryl moiety comprised in said —CO—NH—SO-aryl, said —CO—NH—SO₂-aryl, said —CO—NH—SO—(C₁₋₄ alkylene)aryl, or said —CO—NH—SO₂—(C₁₋₄ alkylene)aryl is optionally substituted with one or more groups independently selected from C₁₋₄ alkyl, halogen, —CF₃, —CN, —NO₂, —N₃, —OH, —O(C₁₋₄ alkyl), —SH, —S(C₁₋₄ alkyl), —NH₂, —NH(C₁₋₄ alkyl), or —N(C₁₋₄ alkyl)(C₁₋₄ alkyl), and further wherein the heteroaryl moiety comprised in said —CO—NH—SO-heteroaryl, said —CO—NH—SO₂-heteroaryl, said —CO—NH—SO—(C₁₋₄ alkylene)heteroaryl, or said —CO—NH—SO₂—(C₁₋₄ alkylene)heteroaryl is optionally substituted with one or more groups independently selected from C₁₋₄ alkyl, halogen, —CF₃, —CN, —NO₂, —N₃, —OH, —O(C₁₋₄ alkyl), —SH, —S(C₁₋₄ alkyl), —NH₂, —NH(C₁₋₄ alkyl), or —N(C₁₋₄ alkyl)(C₁₋₄ alkyl);
L¹¹ is C₁₋₆ alkylene or C₂₋₆ alkenylene, wherein said alkylene or said alkenylene is optionally substituted with one or more groups independently selected from halogen, —CF₃, —CN, —OH, —O(C₁₋₄ alkyl), —NH₂, —NH(C₁₋₄ alkyl), or —N(C₁₋₄ alkyl)(C₁₋₄ alkyl), and further wherein one —CH₂— unit comprised in said alkylene or said alkenylene is optionally replaced by C₃₋₇ cycloalkylene;
L¹² is C₁₋₆ alkylene or C₂₋₆ alkenylene, wherein said alkylene or said alkenylene is optionally substituted with one or more groups independently selected from halogen, —CF₃, —CN, —OH, —O(C₁₋₄ alkyl), —NH₂, —NH(C₁₋₄ alkyl), or —N(C₁₋₄ alkyl)(C₁₋₄ alkyl);
L¹³ is C₁₋₆ alkylene or C₂₋₆ alkenylene, wherein said alkylene or said alkenylene is optionally substituted with one or more groups independently selected from halogen, —CF₃, —CN, —OH, —O(C₁₋₄ alkyl), —NH₂, —NH(C₁₋₄ alkyl), or —N(C₁₋₄ alkyl)(C₁₋₄ alkyl);
n is an integer of 0 to 4; and
m is an integer of 0 to 4;
   or a pharmaceutically acceptable salt or solvate thereof.
7. The pharmaceutical composition of item 6, wherein each R¹¹ is independently selected from halogen, —CF₃, or —CN.
8. The pharmaceutical composition of item 6 or 7, wherein R¹³ is selected from —COOH, tetrazolyl, —SO₃H, —SO—NH₂, —SO₂—NH₂, —CO—NH—OH, or —CO—NH—CN.
9. The pharmaceutical composition of any of items 6 to 8, wherein R¹³ is —COOH.
10. The pharmaceutical composition of any of items 6 to 9, wherein L¹¹ is C₁₋₄ alkylene, wherein one —CH₂— unit comprised in said C₁₋₄ alkylene is replaced by 1,1-cyclopropylene.
11. The pharmaceutical composition of any of items 6 to 10, wherein L¹² is —CH₂CH₂— or —CH₂CH₂CH₂—.
12. The pharmaceutical composition of any of items 6 to 11, wherein L¹³ is C₁₋₄ alkylene.
13. The pharmaceutical composition of any of items 6 to 12, wherein n is 1.
14. The pharmaceutical composition of any of items 6 to 13, wherein m is 0.
15. The pharmaceutical composition of item 14, wherein the leukotriene antagonist or leukotriene receptor antagonist is a compound having the following formula:

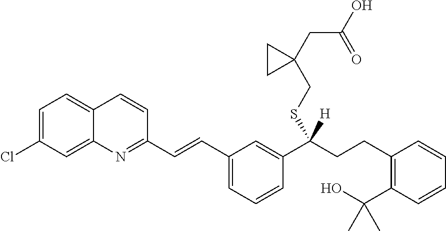 [SMILES: CC(C)(O)c1ccccc1CC[C@@H](SCC1(CC(=O)O)CC1)c1cccc(/C=C/c2ccc3ccc(Cl)cc3n2)c1]

or a pharmaceutically acceptable salt or solvate thereof.
16. A pharmaceutical composition comprising montelukast for use in the treatment of a subject suffering from dementia in Parkinson's disease and/or for use in retrieval or improvement of learning processes or learning deficits and/or the prevention, retrieval or improvement of memory loss or memory impairment in a subject suffering from Parkinson's disease.
17. A pharmaceutical composition comprising montelukast for use in the treatment of a subject suffering from dementia in Parkinson's disease.
18. A pharmaceutical composition comprising montelukast for use in the retrieval or improvement of learning processes or learning deficits in a subject suffering from Parkinson's disease.
19. A pharmaceutical composition comprising montelukast for use in the prevention, retrieval or improvement of memory loss or memory impairment in a subject suffering from Parkinson's disease.

The present invention is also illustrated by the following figures and examples:

The figures show:

FIG. 1: Experimental setup. Montelukast (10 mg kg⁻¹ body weight) or vehicle solution were administered per oral gavage daily for a total period of 42 days. On days 12-15, all rats received i.p. injections of BrdU (50 mg kg⁻¹ body weight). From day 28-41, several behavioral tests were undertaken. Animals were finally perfused on day 42 and processed for histological analysis.

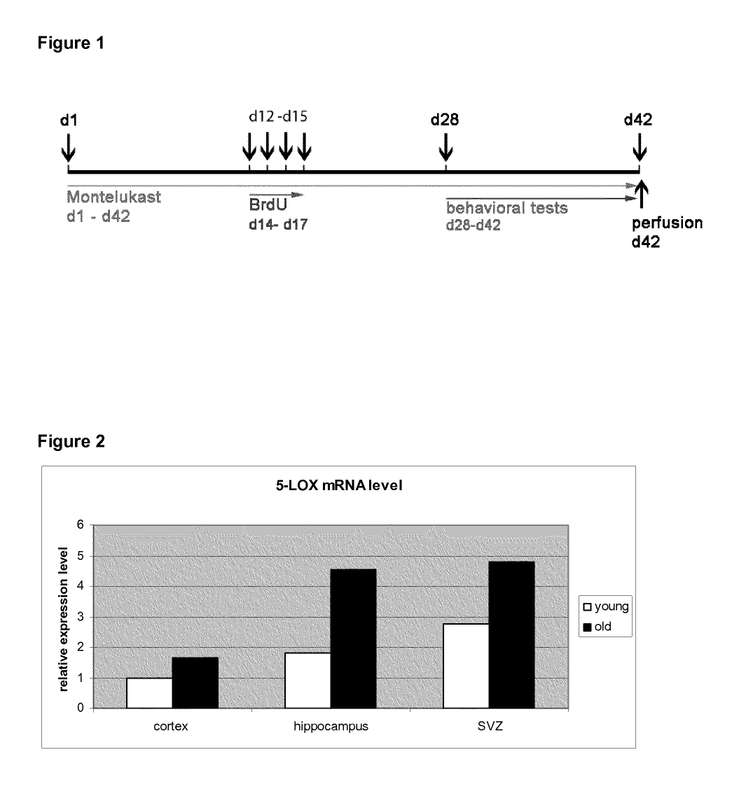

FIG. 2: mRNA expression levels of the 5-Lipoxygenase in young (2 months) and old (20 months) rats. In the neurogenic (hippocampus, SVZ) regions and to a lesser extent in the cortex, the 5-LOX levels were elevated in old compared to young animals.

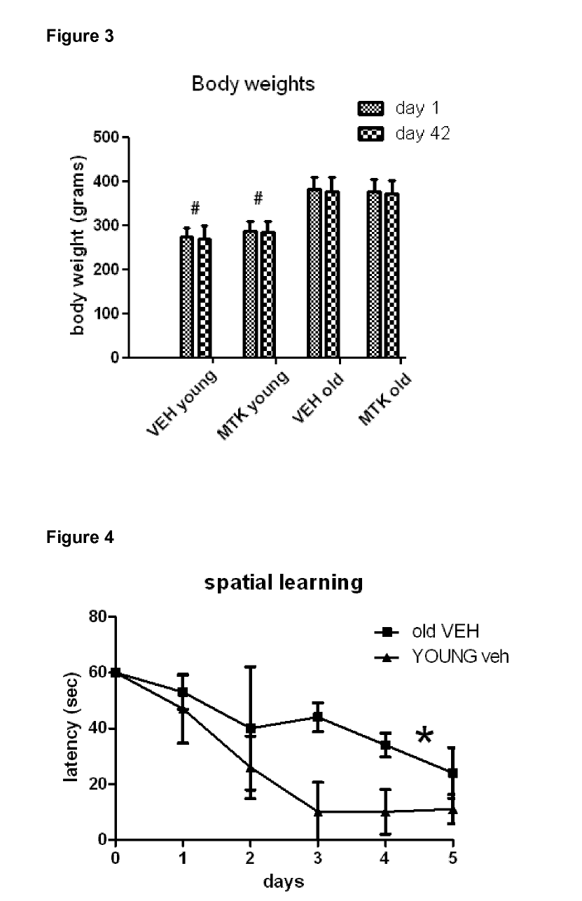

FIG. 3: To detect whether treatment with montelukast causes changes in body weight, the weight of every animal was measured on the first day of montelukast administration (day 1) and 6 weeks later on the last day of administration (day 42). All weights remained stable and represented normal, age-appropriate body weights of F344 rats. Significant differences were only found between the young and old groups. # indicates significant differences (p<0.5) compared to both the old Montelukast and the old vehicle group.

FIG. 4: Impact of aging on the Morris Watermaze learning paradigm. According to their learning curve, old rats were significantly impaired compared to young rats (p<0.01).

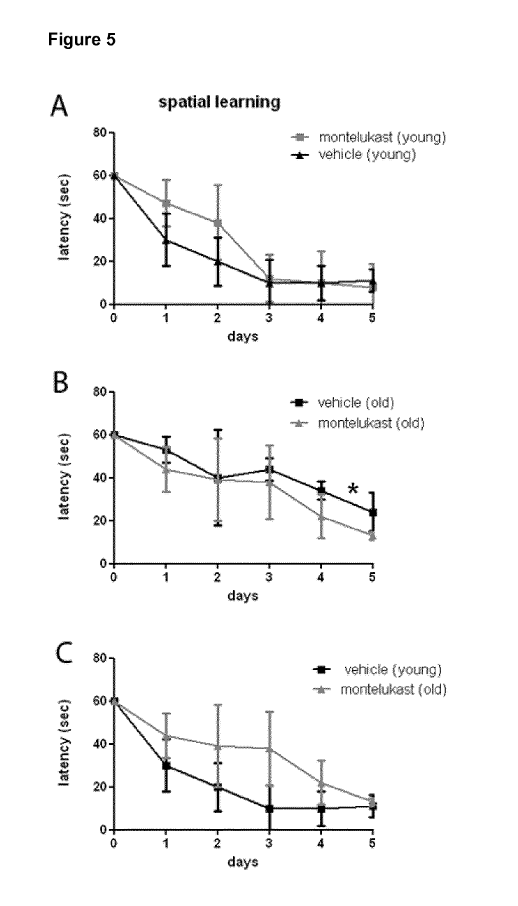

FIG. 5: Morris Watermaze paradigm—spatial learning. Montelukast had no effect on the learning performance of young rats (A), but significantly improved spatial learning capacity in the aged animals (p<0.05) (B). At the end of the training period (day 5), learning capacity of old Montelukast treated rats reached a level comparable with young rats (C).

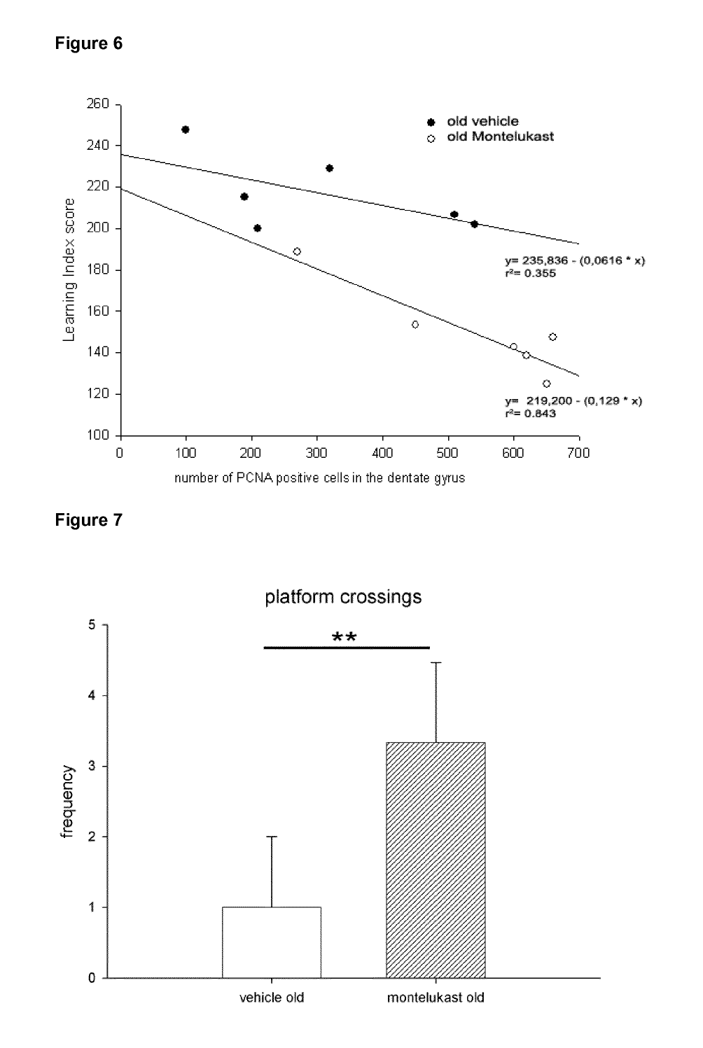

FIG. 6: Montelukast improves the correlation between learning performance and the rate of neurogenesis in old rats. In old montelukast treated animals, the effect of neurogenesis on spatial learning improvement is significantly stronger than in the old vehicle rats. This increased efficiency is indicated by the significant higher slope value of the regression line through the data points of old montelukast treated rats (b=0.129) compared to their vehicle counterparts (b=0.0616). A lower learning index indicates a better learning performance.

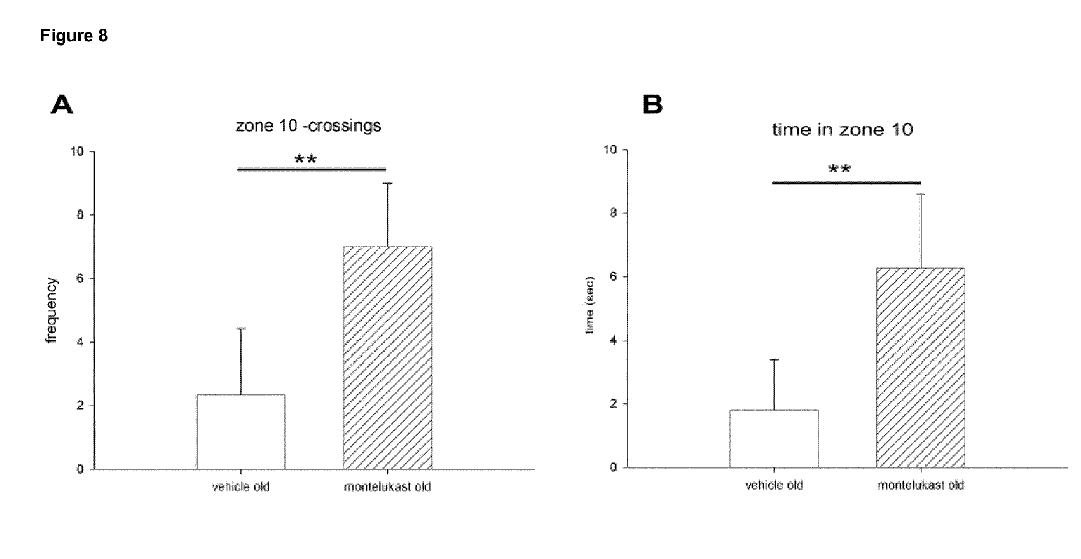

FIG. 7: Morris Watermaze paradigm—memory. Montelukast significantly improved memory in old rats. In the 60 sec probe trial on day 6, old Montelukast treated rats crossed the area of the former platform location significantly more often than old vehicle treated rats.

FIG. 8: Morris Watermaze paradigm—memory. Montelukast significantly improved memory in old rats. During the 60 sec probe trial on day 6, aged Montelukast treated rats crossed the circular zone in closest vicinity to the former platform location (zone 10, 20 cm diameter) significantly more often than old vehicle treated rats (A), and spent significantly more time searching for the platform within this zone (B) than their vehicle-treated counterparts.

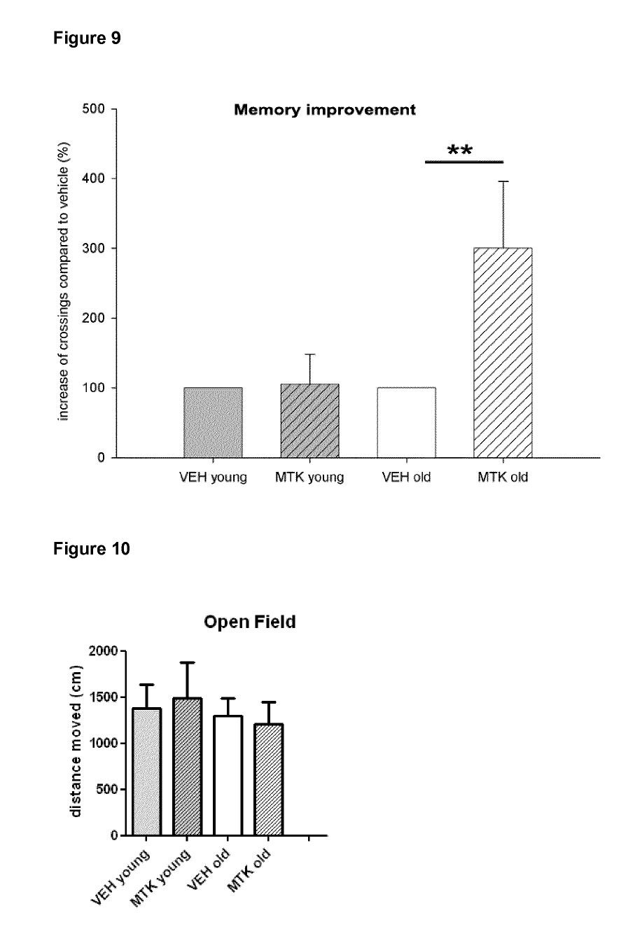

FIG. 9: Morris Watermaze paradigm—memory. Montelukast significantly improved memory in old rats. During the 60 sec probe trial on day 6, old Montelukast treated rats crossed the circular zone around the former platform location (zone 10, 20 cm diameter) significantly more often compared to old vehicle treated rats. In young rats, Montelukast did not alter memory performance.

FIG. 10: Open field test. Administration of Montelukast did not alter general spontaneous locomotor activity in the test animals.

FIG. 11: Forced swim test. Montelukast treatment had no influence on immobility time in young and aged rats.

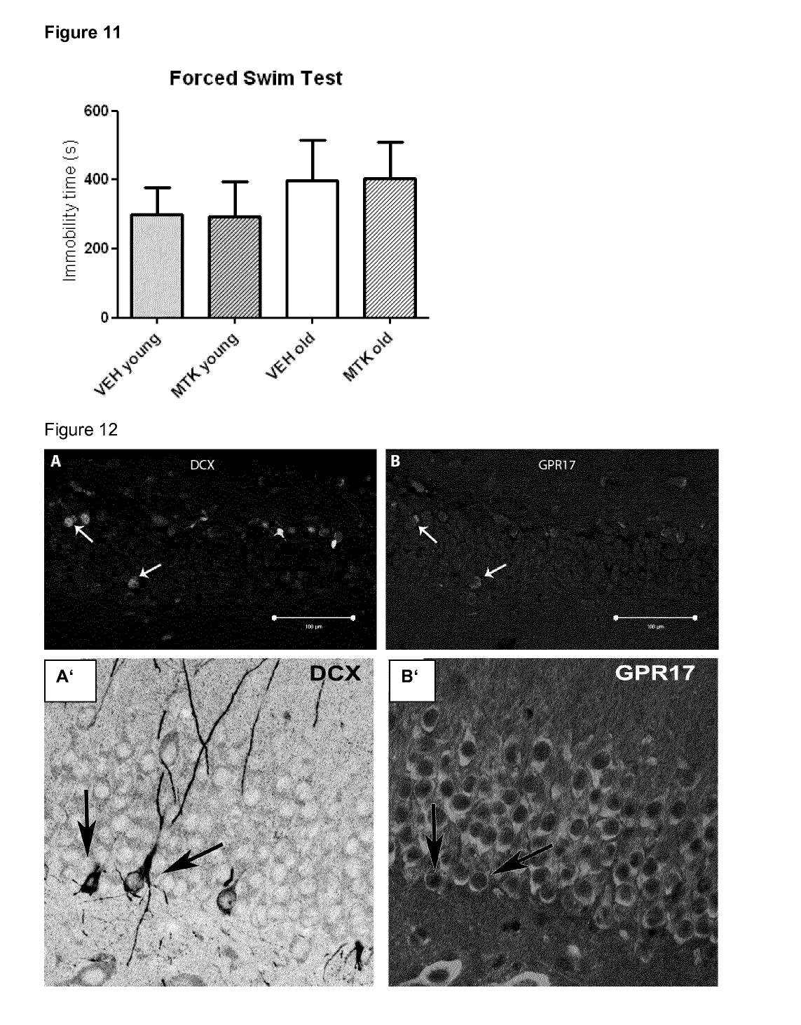

FIG. 12: Immunolabeling of the dentate gyrus with DCX and GPR17. Co-labelling indicates that the GPR17 receptor is expressed by DCX⁺ neuronal precursors cells (arrows).

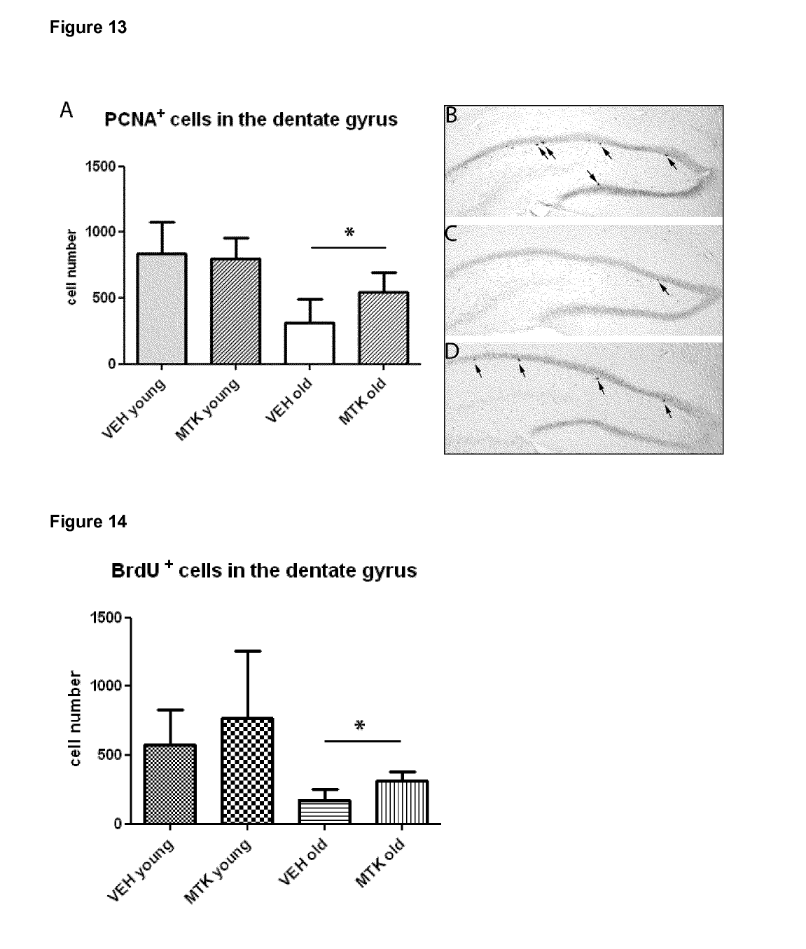

FIG. 13: Montelukast treatment increases cell proliferation in the dentate gyrus of old rats. (A) Absolute number of PCNA positive cells was significantly increased in old Montelukast treated animals compared to the old vehicle group (p<0.05). (B,C,D) Detection of PCNA labelled cells in the dentate gyrus of rats of the young vehicle group (B), the old vehicle group (C) and the old Montelukast group (D).

FIG. 14: Montelukast treatment increases cell survival in the dentate gyrus of old rats. Absolute numbers of BrdU positive cells were significantly increased in old Montelukast treated animals compared to the old vehicle group (p<0.05).

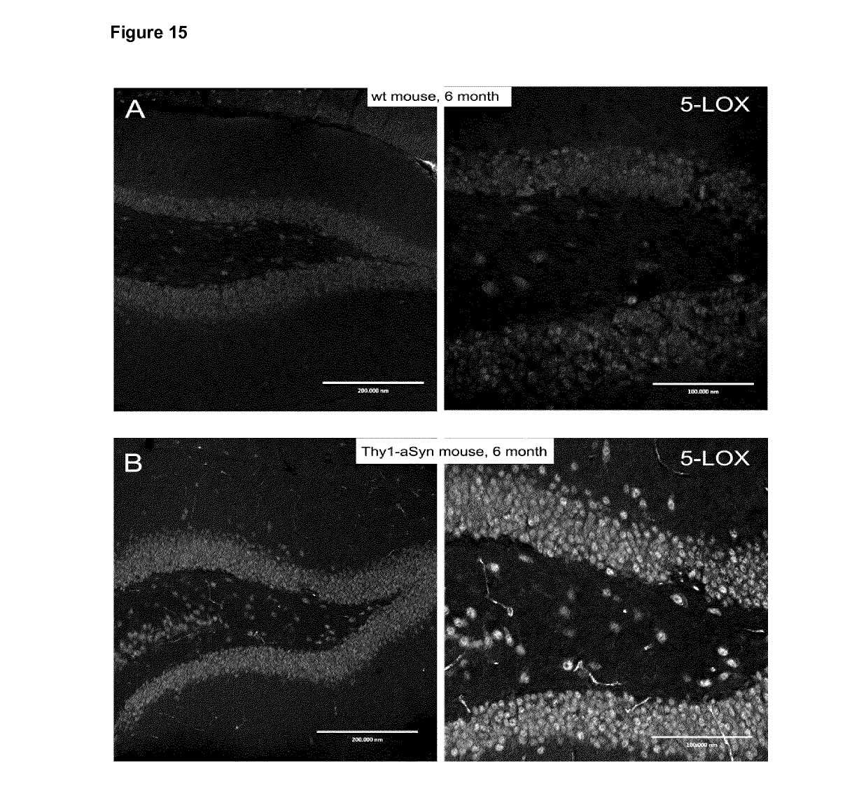

FIG. 15: Elevated expression of 5-LOX protein in the hippocampus of a mouse model of Parkinson's disease (Thy1-aSyn mouse, see, inter alia: Rockenstein, 2002, loc. cit.; Fleming & Chesselet, 2006, loc. cit.; Wu 2010, loc. cit.; Lam, 2011, loc. cit; Chesselet & Richter, 2011, loc. cit; Cabeza-Arvelaiz, 2011, loc. cit.; Fleming, 2006, loc. cit. as well as Magen, 2012, loc. cit. wherein cognitive deficits are documented for this PS mouse model). While 5-LOX immunoreactivity was very faint in the dentate gyrus of the 6 month old wildtype mice (A), it was remarkably more pronounced in 6 month old Thy1-aSyn mice (B).

The present invention is additionally described by way of the following illustrative non-limiting examples that provide a better understanding of the present invention and of its many advantages. The following examples are included to demonstrate preferred embodiments of the invention. It should be appreciated by those of skill in the art that the techniques disclosed in the examples which follow represent techniques used in the present invention to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

### Materials and Methods as Applied in Tests to Proof the Capability of Leukotriene Antagonists to Retrieve or Improve Learning Processes or Learning Deficits and/or to Retrieve or Improve Memory Loss or Memory Impairment

### Animals

Experiments were performed in conformity with the European Community Council Directive and were approved by a national animal health commission. The experiments described in this report were performed on young (4 months) and old (20 months) male Fisher 344 rats bred in the central animal facility of the Paracelsus Medical University, Salzburg, Austria. Said old rats were used as a model system for dementia in Parkinson's disease. They were used in particular as a model system to analyze learning processes or learning deficits and/or the retrieval or improvement of memory loss or memory impairment in dementia in Parkinson's disease.

For RNA isolation and quantitative RT-PCR experiment to detect 5-LOX mRNA levels, young (2 months) and old (20 moths) male Fisher 344 rats have been used. All animals were housed under standard conditions of a 12 hour light/dark cycle with food and water ad libitum. Between 2 and 5 rats were kept in one cage and genders were kept separated.

### RNA Isolation and Quantitative RT-PCR

The expression of 5-LOX at the mRNA level in specific brain regions (hippocampus, subventricular zone (SVZ), cortex) of young (2 months, n=2) and old (20 months, n=2) male F-344 rats has been evaluated by quantitative real time PCR. After decapitation of the animals, the brains were removed and the tissues of interest were isolated.

For isolation of RNA, the brain samples were homogenized in 1 ml of Trizol (TRI® Reagent; Sigma, Taufkirchen). For phase separation, 200 μl of 1-bromo-3-chloropropane were added, vortexed and centrifuged (15 min at 12000 g). After transferring the aqueous phase into a new tube and adding 350 ml of ethanol, RNA extraction was performed using the QIAGEN RNEasy Mini Kit (Qiagen, Hilden, Germany) and cDNA was synthesized using Promega reverse transcription Kit (Promega). Expression analysis was performed by a TaqMan gene expression assays kit (Applied Biosystems, California, USA) for the rat 5-LOX gene (Rn00689111_m1). Probes and primers were provided by manufacturer (Applied Biosystems, California, USA). Rat Glyceraldehyde 3-phosphate dehydrogenase (GAPDH) was used as endogenous control gene. The following temperature profile was used: activation of polymerase 95° C., 10 min; 40 cycles of denaturing 95° C., 15 s, and annealing/extension 60° C., 60 s. Data were obtained with a Rotor-Gene 6000 R Corbett Research (geneXpress, Vienna, Austria) and analyzed by delta delta Ct method (Livak and Schmittgen (2001) Methods 25:402-408).

### Montelukast Administration and BrdU Injections

Montelukast sodium powder (Sigma, Germany) was first dissolved in ethanol for maximum solubility and then further diluted (1:9 ratio) with a 0.9% saline (NaCl) solution. The solution was made fresh every day and administered daily per oral gavage (p.o) at a dose of 10 mg kg⁻¹ of body weight for 42 days (FIG. 1). Control animals received volume-matched injections of the vehicle solution (ethanol:0.9% NaCl) only. Doses for Montelukast were selected on the basis of previous tests in the laboratory, and according to reports in the literature of intraperitoneal Montelukast injections (Biber, 2009, Brain Inj 23(6): 577-584). The rats were divided into four experimental groups as follows: young control group (vehicle p.o.; n=10), young Montelukast group (10 mg kg⁻¹ Montelukast p.o.; n=10), old control group (vehicle p.o.; n=7), old Montelukast group (10 mg kg⁻¹ Montelukast p.o.; n=7).

For the analysis of cell survival and cell differentiation, all rats received intraperitoneal injections of BrdU (Sigma, Germany) at 50 mg kg⁻¹ of body weight dissolved in 0.9% saline solution on day 12, 13, 14 and 15. All animals were weighed on the first day of Montelukast administration and on the last day of the experiment (day 42).

### Behavioral Assessment

28 days after starting Montelukast treatment, several standardized behavioral tests have been carried out. All behavioral experiments were done in a windowless room with the size of 3.5 m×2.7 m×2.2 m and a room temperature of 24° C.±1° C. The door was black colored and on the walls were white tiles. The computer was situated in one corner of the room connected to a camera located in the middle of the ceiling facing down. All parameters analysed were recorded via the use of the video tracking software Ethovision 2.0 (Noldus Information Technology, Wageningen, Netherlands).

### Open Field Test

On day 29 after the first Montelukast administration, the open field test was carried out to detect spontaneous changes in general locomotor activity. The open field was a black circular plastic plate with a diameter of 1 m and a 5 cm high wall. The apparatus was set in the middle of the testing room. Before each trial began, the open field was cleaned with 70% ethanol and permitted to dry between the tests. Each rat was placed into the center of the plate and was allowed to explore the apparatus for 5 minutes. Total distance and average speed were recorded.

### Forced Swim Test

On day 33, the forced swim test was used to analyse depressive-like behavior in the test animals. The rats were forced to swim for 10 min in a square plastic tank (40 cm in diameter) filled to a depth of 30 cm with tap water (20±1° C.). During the forced swimming session, the behavior was recorded by a video system and scored by a trained observer, quantifying absolute time measurements. The behavior of the animals was assigned to one of the three following behavioral categories: (1) struggling, defined as movements during which the forelimbs broke the surface of water; (2) swimming, defined as movement of the animal induced by movements of the fore and hind limbs without breaking the water surface; and (3) immobility time, defined as the behavior during which the animal used limb movement just to keep its equilibrium without any movement of the trunk. After the 10 min swimming session, animals were gently dried using a towel and returned to their home cage.

### Morris Watermaze

On days 35-41, the water-maze procedure was used to assess the ability of spatial learning and memory. The apparatus consisted of a circular swimming pool built of black plastic (170 cm diameter, 30 cm height), filled with 21° C.±1° C. tempered water. The tank was divided into four equal quadrants, with a submerged hidden 10×10 cm fibreglass platform placed 3 cm below the water surface in the middle of the target quadrant. The position of the platform was kept unaltered throughout the learning sessions. In the testing room, several big black cue symbols were put on each wall for spatial orientation. The water maze task was carried out twice a day for 5 consecutive days. One day before starting the learning experiment (day 0), each rat was put into the water and was allowed to locate the submerged platform for 60 seconds. If the animal failed to find the platform within the 60 seconds, it was guided onto the platform and allowed to remain there for ten seconds. For the learning tasks on days 1-5, each rat was put into the water at one of four starting positions, the sequence of which being selected randomly, with each trial having a ceiling time of 60 seconds to find the platform. The escape latency time to locate the hidden platform and the distance moved during the trail was recorded with the camera software as indices of spatial learning. On day 6, after the learning phase of the experiment, a probe trial was performed. Here, the platform was removed and each animal was allowed to explore the pool for 60 seconds. From the probe trial, several parameters were recorded as indices of memory: number of crossings of the former platform location, number of crossings of ‘zone 10’ (a defined circular area with 20 cm diameter around the former platform location) and time spent in zone 10.

### Perfusion and Tissue Processing

On day 42, the animals were deeply anaesthetized using a ketamine (20.38 mg/ml), xylazine (5.38 mg/ml) and acepromazine (0.29 mg/ml) mixture. Transcardial perfusion was performed with 0.9% NaCl solution, followed by a 4% paraformaldehyde, 0.1M sodium phosphate solution (pH 7.4). The brains were dissected and post-fixed in the paraformaldehyde solution overnight at 4° C. Tissues were then cryoprotected in a 30% sucrose, 0.1M sodium phosphate solution (pH 7.4). Brains were cut into 40 μm saggital sections using a sliding microtome on dry ice. Sections were stored at −20° C. in cryoprotectant solution (ethylene glycol, glycerol, 0.1 M phosphate buffer pH 7.4, 1:1:2 by volume).

### Immunohistochemistry

Free-floating sections were treated with 0.6% H₂O₂ in tris-buffered saline (TBS: 0.15M NaCl, 0.1M Tris-HCl, pH 7.5) for 30 min, followed by three washes with TBS. For immunological detection of PCNA, the sections were subjected to the following procedure: incubation in 0.3 M NaCl/30 mM citrate buffer (pH7.0)/50% formamide at 65° C. for 2 hours, rinsed in 0.3M NaCl/30 mM citrate buffer (pH 7.0), incubation in 2 N HCl at 37° C. for 30 minutes, rinsed in 0.1 M borate buffer (pH 8.5) for 10 minutes and rinsed in TBS. Sections were blocked with a solution composed of TBS, 0.1% Triton X-100, 1% bovine serum albumin, and 0.2% teleostean gelatin (Sigma, Taufkirchen, Germany) for 1 hour. This buffer was also used during the incubation with primary antibodies, which were applied overnight at 4° C. For chromogenic immunodetection, the sections were washed extensively and further incubated for 1 hour with a biotin-conjugated species-specific secondary antibody. Sections were then incubated for 1 hour in a peroxidase-avidin complex solution (Vectastain Elite ABC kit; Vector Laboratories, Burlingame, Calif.). The peroxidase activity of immune complexes was revealed with a solution of TBS containing 0.25 mg/mL 3,3 diaminobenzidine (Vector Laboratories), 0.01% H₂O₂, and 0.04% NiCl₂. Sections were placed on Superfrost Plus slides (Menzel, Braunschweig, Germany) and mounted on Neo-Mount (Merck, Darmstadt, Germany). For epifluorescence immunodetection, sections were washed extensively after primary antibody incubation and incubated with fluorochrome-conjugated species-specific secondary antibodies overnight at 4° C. Sections were put on slides and mounted in Prolong Antifade kit (Molecular Probes, Eugene, USA).

The following antibodies and final dilutions were used. Primary antibodies: mouse anti-PCNA 1:500, goat anti-GPR17 1:250 (both Santa Cruz Labs, Santa Cruz, USA), and rabbit DCX 1:500 (Cell Signaling, Danvers, USA). Secondary antibodies: rabbit anti mouse biotinylated 1:500 (Dianova, Hamburg, Germany), donkey anti goat conjugated with Alexa 488 1:1000, and donkey anti goat conjugated with Alexa 568 1:1000 (both Molecular Probes, Eugene, USA).

Photodocumentation was done using a Zeiss Axioplan microscope equipped with the Zeiss AxioVision imaging system, and epifluorescence observation was performed on a confocal scanning laser microscope (CLSM 510 META; Zeiss, Goettingen, Germany) with LSM software.

### Counting Procedures

For quantification of PCNA positive cells in the dentate gyrus, analyses were performed blinded on coded slides. Every tenth section (400 μm interval) of one hemisphere was selected from each animal and processed for immunhistochemistry. To analyse cell proliferation in the dentate gyrus, PCNA-positive cells were counted on a Zeiss Axioplan microscope and multiplied by 10 to obtain an estimate of total immunopositive cell numbers. The reference volume of the dentate gyrus was determined by tracing the area on each analysed section.

### Statistical Analysis

Statistical analyses were performed using the GraphPad Prism 4.0 software (GraphPad Software, San Diego, Calif., USA). Data were analysed by one way analysis of variance (ANOVA) or two way ANOVA, followed by Bonferroni post hoc tests when necessary. The ‘Pearson Product Moment Correlation test’ was performed to assess correlation between variables. P values <0.05 were considered to be statistically significant. All values were expressed as means±standard deviation.

### EXAMPLE 2

### 5-LOX mRNA Expression is Increased in Old Rats Predominantly in Neurogenic Regions

5-lipoxygenase (5-LOX) is the rate-liming enzyme that catalyzes the production of leukotrienes, and the level of 5-LOX mRNA expression is generally used as an indirect measure of 5-LOX activity. Following the hypothesis that age-related reduction in cognitive performance and in neurogenesis might be due to elevated levels of leukotrienes present in the aged neurogenic niches, 5-LOX mRNA expression levels were analyzed in different brain regions of young and old rats applying quantitative real time PCR. Expression of 5-LOX mRNA was notably increased within the neurogenic regions (hippocampus: 2.4 fold; SVZ: 1.8 fold) of aged rats compared to the young animals. 5-LOX expression was also elevated in the cortex (1.7 fold) of old rats compared to the cortex of young rats, but to a lesser extend as in the hippocampus or SVZ (FIG. 2).

### EXAMPLE 3

### Montelukast Treatment does not Alter Body Weight

GPR17 is highly expressed in food-intake-regulating Agrp Neurons, and intraventricular infusion of GPR17 agonists elevates food intake (Ren, 2012, Cell, 8; 149(6):1314-26). Therefore, it was tested whether the oral application of Montelukast had any side effects on body weight. Therefore, each rat was weighed at the beginning of the experiment (day 1) and directly before the perfusion (day 42). Old rats were significantly heavier than young rats, but the body weights of all rats remained constant over the period of the experiment. The Montelukast treated groups did not differ in their body weight compared to their vehicle treated counterparts (FIG. 3).

### EXAMPLE 4

### GPR17 is Expressed in DCX Positive Cells in the Dentate Gyrus

To obtain information about the cell types affected by the GPR17 antagonist Montelukast, immunofluorsecent staining against the GPR17 receptor was performed. Co-labelling of GPR17 with DCX (marker for neuronal precursors) within the dentate gyrus of rats indicated the expression of GPR17 by hippocampal neuronal precursor cells (FIG. 12).

### EXAMPLE 5

### Montelukast Treatment Increases Cell Proliferation and Cell Survival in the Dentate Gyrus of Old Rats

To assess possible changes in the proliferative activity within the hippocampal neurogenic niche after Montelukast administration, cell proliferation was analysed by quantifying PCNA positive cells within the dentate gyrus. PCNA, a marker detected in the nucleus of proliferating cells, is routinely used as a proliferation marker for adult hippocampal neurogenesis (von Bohlen, 2011, Cell Tissue Res., 345, 1-19).

Possible effects of Montelukast treatment on the survival rate of newly generated cells within the dentate gyrus were evaluated by using the BrdU cell survival paradigm. 4 weeks before perfusion, all animals received intraperitoneal injections of BrdU (see FIG. 1), a thymidine analogue that incorporates into dividing cells. Hence, quantification of BrdU positive cells within the dentate gyrus on histological tissue reflects the number of cells that were newly generated 4 weeks before perfusion and survived until the end of the experiment.

The PCNA data revealed a significant increase in cell proliferation in the dentate gyrus of old (20 months) montelukast treated rats, compared to the old vehicle treated rats. No significant differences could be detected between the young (4 months) treatment groups (FIG. 13). Similarly, numbers of BrdU positive cells within the dentate gyrus, reflecting the survival rate of newly generated cells, were significantly increased in the old montelukast treated rats compared to the old vehicle group. Again, no differences were detected between the young groups (FIG. 14).

These results for the first time illustrate that montelukast increases cell proliferation and cell survival within the hippocampus in vivo. The present in vivo data together with previous work, where we have demonstrated that montelukast increases the proliferation of hippocampal neural progenitor cells of young adult rats in vitro (Huber, 2011, Cell. Physiol. Biochem., 28, 793-804), let strongly assume that boosting the rate of neurogenesis is a major mode of action of montelukast.

However, surprisingly, montelukast treatment in vivo affected cell proliferation exclusively in old animals. This astonishing age-dependent effect of montelukast could not be anticipated, since in general young adult rodents, which possess a several fold higher rate of neurogenesis, are more susceptible to modulation of neurogenesis than aged rodents, in which neurogenesis has already considerably decreased (reviewed in Lazarov, 2010, Trends Neurosci., 33, 569-579). Thus, changes in the level of adult neurogenesis are typically only achieved in young adult animals (Couillard-Despres, 2009, Mol. Psychiatry, 14, 856-864; Gil-Mohapel, 2010, Eur. J. Neurosci., 31, 797-807) or in both young and aged animals (van Praag, 2005, J. Neurosci., 25, 8680-8685), but are not expected to be related exclusively to old animals. Further, in vitro data showing a proliferative effect of montelukast in neural progenitor cells obtained from young adult (2-4 months) rats, would rather let expect an in vivo effect in young adult rats than in old animals.

### EXAMPLE 6

### Oral Treatment with Montelukast Improves Learning and Memory Abilites in Aged Rats without Inducing Depression-Like Behavior or Altering General Locomotor Activity

By means of the Morris Watermaze task, it was examined if blocking the leukotriene signaling pathway by Montelukast could revert the cognitive weaknesses of old rats. The Morris Watermaze task, originally invented by Richard Morris (Morris, 1981, Learn Motiv, 12, 239-260), has become one of the most frequently used paradigms to evaluate learning and memory in rodents (D'Hooge, 2001, Brain Res. Brain Res. Rev, 36, 60-90). In this spatial navigation task, in which the animals swim to find a hidden platform, distant visual landmarks in the environment are used to locate the platform. Escape from the water is the positive reinforcement, and the task is based on the principle that rodents are highly motivated to escape from a water environment by the quickest, direct route. It has been shown that hippopcampal lesions and pharmacological treatments impair acquisition of the Morris Water Maze task (Morris (1981) Learn Motiv, 12, 239-260; Nilsson, 1993, Neurobiol Aging, 14, 487-497). Further, rodent models of neurodegenerative disorders (e.g Chen et al., 2000), but also ‘healthy aged’ rats (Gallagher et al. (1993) Behav Neurosci 107:618-626; Bizon, 2009, Neurobiol. Aging, 30, 646-655) show dramatic impairments in performance on this task.

### Spatial Learning Performance was Impaired in Old Compared to Young Rats

Analysis of the young and old vehicle-treated rats revealed, as expected (Gallagher et al. (1993) Behav Neurosci 107:618-626; Bizon, 2009, Neurobiol. Aging, 30, 646-655), a statistically significant weakness in the learning performance of aged compared to young rats (FIG. 4). Old animals reached the hidden platform after a significant longer latency period, indicating impaired spatial learning.

### Montelukast Improves Spatial Learning in Old Rats

Effects of montelukast administration on spatial learning behavior have been examined. The Morris Watermaze paradigm revealed that Montelukast treatment had no effects on the learning performance of young (4 months) rats (FIG. 5A). However, in old (20 months) rats, montelukast remarkably improved spatial learning (FIG. 5B). The learning curve over the training days 1-5 was significantly enhanced in the old montelukast treated rats compared to the old vehicle animals, in that the latency to find the hidden platform was significantly lower in the old montelukast treated animals. Reduced latency to find the hidden platform further reflects more successful learning of the platform location and constitutes the main index to measure spatial learning in the morris watermaze task.

An additional surprising finding was an approximation of the learning curve of the old Montelukast treated animals to the learning curve of young rats over time. We surprisingly found that at the end of the training period (day 5), the old Montelukast treated animals were as successful as young rats in finding the hidden platform (FIG. 5C). These data demonstrate that administration of montelukast not only causes some significant improvement of the learning capacity in old rats, but moreover seems to lead to a full recovery of the age-related learning deficiencies.

Next, by plotting the individual learning scores of each animal (calculated from the spatial learning data obtained by the watermaze paradigm) against its rate of neurogenesis (number of PCNA positive cells in the dentate gyrus), we could demonstrate that learning performance indeed positively correlates with the rate of neurogenesis in old rats, although in the literature an inversed correlation between the level of neurogenesis and cognitive performance is claimed for old rodents (Bizon et al. (2004) Aging cell 3:227-234; Lazic (2010) Neurobiol Aging 31:2169-2171) (FIG. 6). Excitingly, montelukast treatment in old rats further leads to an increase of the effect of neurogenesis on spatial learning improvement. The montelukast treated animals exhibit elevated efficiency of the individual neurogenesis rate to improve spatial learning, as indicated by a higher slope of the regression line for montelukast treated rats than for their untreated counterparts (FIG. 6). Thus, even similar amounts of neurogenesis may lead to better learning output in old montelukast treated rats compared to the vehicle group.

The age-specific improvement of spatial learning performance, observed exclusively in old animals, represents a most intriguing novel finding. When utilizing the standard, routinely used approach of studying neurogenesis and cognitive changes only in young rats, the fascinating effects of montelukast on aged animals would have been easily missed out.

In the field of adult neurogenesis research, it is commonly accepted to study adult neurogenesis in young animals. From 18 months onwards, rats are usually designated as old (aged) animals (Frick et al (1995) Neurobiol Aging 16:149-160, Bizon (2009) Neurobiol. Aging, 30, 646-655). Working with the young age cohorts bears several practical and sometimes also scientific advantages. Young rats are more rapid and easier available for experiments than old animals, since rearing time is considerably shorter. Likewise, the costs to rear rats from birth to the young adult stage (about 4 months) are considerably lower than rearing rats until they reach senescence (about 20 months). In a scientific view, modulation of neurogenesis is generally easier and more successful in young rodents that already possess a high level of neurogenesis, than in old animals, in which the pool of neural stem cells is already exhausted and/or stem cells have retired to a quiescent state (Lazarov, 2010, Trends Neurosci., 33, 569-579). In conformity with the more promising modulation of neurogenesis in young animals, attempts to improve cognitive abilities are similarly more successful in young than in old animals (e.g. Asl, 2008, Pathophysiology, 15, 9-12). Beyond these reasons for using young animals to study effects on neurogenesis and cognition, the montelukast in vitro data would furthermore let expect, if at all, only in vivo effects of montelukast in young rats (Huber, 2011, Cell. Physiol. Biochem., 28, 793-804). Due to practical limitations, Huber et al. (supra) were restricted to test the effects of montelukast in primary neurospheres from young (2-4 months old) rats, since neurospheres of middle aged and old rats exhibit impairments in their in vitro properties (Bouab, 2011, Neuroscience, 173, 135-149).

Thus, in respect to the present knowledge, increasing the spatial learning capacity exclusively in old animals via montelukast constitutes a surprising, innovative finding that could not be anticipated.

### Memory in Old Rats is Significantly Improved by Montelukast

To test if Montelukast improved memory skills, the platform was removed on day 6 and the following parameters were analyzed: number of crossings of the former platform location; number of crossings of zone 10 (20 cm area around the former platform location); time spent in zone 10.

FIG. 7 illustrates that Montelukast treated aged rats performed significantly more platform crossings compared to the vehicle treated animals indicating enhanced memory function in the Montelukast treated animals.

A similar observation was made, when the area of crossings was enlarged to a 20 cm area around the former platform (zone 10). Also, here, the Montelukast treated aged rats expressed a significantly better memory compared to the vehicle treated group (FIG. 8A). Moreover, the analysis of time spent in zone 10 clearly showed that Montelukast treated aged rats had a higher memory function compared to vehicle treated controls (FIG. 8B).

To compare the memory enhancing effects of Montelukast in aged versus young animals, the memory capacity (zone 10 crossings at day 6) of Montelukast treated animals was normalized for vehicle treated animals, which was set as 100%. FIG. 9 illustrates that Montelukast enhanced memory specifically in aged rats, but not in young animals.

As for the spatial learning results (see above), this age-specific effect, showing that montelukast improves memory abilities exclusively in old rats, constitutes a novel, innovative finding. Achieving memory improvements entirely in old individuals, for which a recovery of the cognitive deficits is essential and highly desired, has, to our knowledge, not been demonstrated so far.

Montelukast does not Alter General Locomotor Activity Nor does it Induce Depression-Like Behavior

The ‘open field’ test to detect spontaneous changes in general locomotor activity revealed no significant changes between the groups (FIG. 10). Further, the immobility time detected by the forced swim test was not significantly different between any of the tested groups, indicating that treatment with Montelukast did not cause depressant-like behavior (FIG. 10).

### EXAMPLE 7

Further support for the inventive use of a leukotriene antagonist or a leukotriene receptor antagonist, in particular Montelukast, in Parkinson's dementia and/or in the amelioration of learning deficiencies in Parkinson's patients/Parkinson's patients suffering from dementia is provided in the following:

As documented for the first time herein and in the following 5-Lipoxygenase (5-LOX), the key enzyme in leukotriene synthesis, is remarkably and surprisingly elevated in the hippocampus of 6 month-old Thy1-aSyn mice, compared to age-matched wildtype mice (see also FIG. 15) This transgenic line, expressing human wildtype alpha synuclein under the Thy1-promotor, serves as a convincing model of Parkinson's disease (Rockenstein et al., 2002). Thy1-aSyn mice display widespread over-expression of alpha-synuclein in neurons in the brain, progressive synaptic anomalies and loss of striatal dopamine (Fleming & Chesselet, 2006; Wu et al. 2010; Lam et al., 2011; Chesselet & Richter, 2011). A transcriptome analyses revealed alterations in the expression of neurogenesis-related genes, indicating an impairment of adult neurogenesis in this transgenic mouse model (Cabeza-Arvelaiz et al., 2012). These mice further exhibit robust motor dysfunctions (Fleming et al., 2006) and cognitive deficits from 4 month of age on (Magen et al., 2012). The corresponding literature to and information on Thy1-aSyn mice can be found, inter alia, in: Cabeza-Arvelaiz (2012), PLoS One 7(9):e44700; Chesselet (2011), Lancet Neurol. 2; 10:1108-1118; Fleming (2006), Neuroscience 142(4):1245-53.; Lam (2011), J Neurosci Res. 89:1091-1102; Magen (2012), Eur J Neurosci 35:870-882; Rockenstein (2002), J Neurosci Res; 68:568-578; Wu. 2010; J Neurosci Res. 88:1764-1776.

The increased expression of 5-LOX, the enzyme stimulating leukotriene synthesis, in the hippocampus of Thy1-aSyn mice (see, inter alia: Rockenstein, 2002, loc. cit.; Fleming & Chesselet, 2006, loc. cit.; Wu 2010, loc. cit.; Lam, 2011, loc. cit; Chesselet & Richter, 2011, loc. cit; Cabeza-Arvelaiz, 2011, loc. cit.; Fleming, 2006, loc. cit. as well as Magen, 2012, loc. cit. wherein cognitive deficits are documented for this PS mouse model) plausibly documents that dementias in Parkinson's patients can be positively influenced by a leukotriene antagonist or a leukotriene receptor antagonist, like Montelukast. The inhibition of leukotriene signaling by leukotriene receptor antagonists in the hippocampus of mouse models of Parkinson's makes it furthermore plausible that Parkinson's disease dementia can successfully be treated by such leukotriene antagonists or leukotriene receptor antagonists. It is now also feasible to treat Parkinson's disease related cognitive deficits and learning deficiencies.

A strong increase of 5-Lox expression has been reported in the hippocampus of patients suffering of Alzheimer's disease (AD), as well as in AD transgenic mouse models. The hippocampus, which is involved in the cognitive processing, is severely affected by the AD pathological processes. The increase of 5-Lox expression leads to a local increase in leukotriene concentrations. Surprisingly, it is observed herein that 5-Lox expression is also increased in the hippocampus of the Thy1-aSyn mouse (i.e. a non-human Parkinson's model that also shows cognitive deficits from 4 month of age and is, accordingly, a model for Parkinson's dementias), although the hippocampus is not a primary target of this disease. Hence, these unexpected findings suggest that the medical use of leukotriene receptor antagonists, such as Montelukast, constitutes a valid intervention in the alleviation of cognitive impairments of Parkinson's disease, atypical forms of Parkinson's disease/Parkinson's dementia as well in individuals suffering from or prone to suffer from Parkinson's Disease Dementia, Lewy Body Dementia and/or Multi-Systems-Atrophy.

1. A pharmaceutical composition comprising a leukotriene antagonist or a leukotriene receptor antagonist for use in the treatment of a subject suffering from dementia in Parkinson's disease.

2. A pharmaceutical composition comprising a leukotriene antagonist or a leukotriene receptor antagonist for use in retrieval or improvement of learning processes or learning deficits and/or the prevention, retrieval or improvement of memory loss or memory impairment in a subject suffering from Parkinson's disease.

3. The pharmaceutical composition of claim 2, wherein the subject is a subject suffering from dementia in Parkinson's disease.

4. The pharmaceutical composition of any of claims 1 to 3, wherein the subject is a human person.

5. The pharmaceutical composition of claim 4, wherein the human person is at least 20 years old.

6. The pharmaceutical composition of any of claims 1 to 5, wherein the pharmaceutical composition comprises the leukotriene antagonist or the leukotriene receptor antagonist as the sole active ingredient.

7. The pharmaceutical composition of any of claims 1 to 6, wherein the pharmaceutical composition is administered as a monotherapy.

8. A method for the treatment of dementia in Parkinson's disease comprising the step of administering a leukotriene antagonist or a leukotriene receptor antagonist to a subject in need thereof.

9. A method for retrieval or improvement of learning processes or learning deficits and/or the prevention, retrieval or improvement of memory loss or memory impairment in a subject suffering from Parkinson's disease comprising the step of administering a leukotriene antagonist or a leukotriene receptor antagonist to a subject in need of such treatment.

10. The method of claim 9, wherein the subject is a subject suffering from dementia in Parkinson's disease.

11. The method of any of claims 8 to 10, wherein the subject is a human person.

12. The method of claim 11, wherein the human person is at least 20 years old.

13. The method of any of claims 8 to 12, wherein the leukotriene antagonist or leukotriene receptor antagonist is administered as the sole active ingredient of a pharmaceutical composition.

14. The method of any of claims 8 to 13, wherein the leukotriene antagonist or leukotriene receptor antagonist is administered in monotherapy.

15. A leukotriene antagonist or a leukotriene receptor antagonist for use in the treatment of a subject suffering from dementia in Parkinson's disease.

16. A leukotriene antagonist or a leukotriene receptor antagonist for use in the retrieval or improvement of learning processes or learning deficits and/or the prevention, retrieval or improvement of memory loss or memory impairment in a subject suffering from Parkinson's disease.

17. The leukotriene antagonist or the leukotriene receptor antagonist for use according to claim 16, wherein the subject is a subject suffering from dementia in Parkinson's disease.

18. The leukotriene antagonist or the leukotriene receptor antagonist for use according to any of claims 15 to 17, wherein the subject is a human person.

19. The leukotriene antagonist or the leukotriene receptor antagonist for use according to claim 18, wherein the human person is at least 20 years old.

20. The leukotriene antagonist or the leukotriene receptor antagonist for use according to any of claims 15 to 19, wherein the leukotriene antagonist or the leukotriene receptor antagonist is comprised in a medicament as the sole active ingredient.

21. The leukotriene antagonist or the leukotriene receptor antagonist for use according to any of claims 15 to 21, wherein the leukotriene antagonist or the leukotriene receptor antagonist is administered as a monotherapy.

22. The pharmaceutical composition of any of claims 1 to 7 or the method of any of claims 8 to 14 or the leukotriene antagonist or the leukotriene receptor antagonist for use according to any of claims 15 to 21, wherein the leukotriene antagonist or leukotriene receptor antagonist is a compound of the following formula (I): 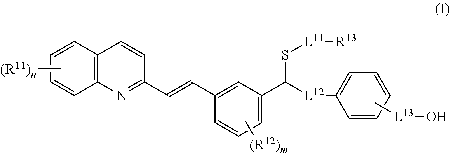
wherein:
each R¹¹ is independently selected from halogen, —CF₃, —CN, —NO₂, —N₃, C₁₋₄ alkyl, —OH, —O(C₁₋₄ alkyl), —SH, —S(C₁₋₄ alkyl), —NH₂, —NH(C₁₋₄ alkyl), or —N(C₁₋₄ alkyl)(C₁₋₄ alkyl);
each R¹² is independently selected from C₁₋₄ alkyl, halogen, —CF₃, —CN, —NO₂, —N₃, —OH, —O(C₁₋₄ alkyl), —SH, —S(C₁₋₄ alkyl), —NH₂, —NH(C₁₋₄ alkyl), or —N(C₁₋₄ alkyl)(C₁₋₄ alkyl);
R¹³ is selected from —COOH, tetrazolyl, —SO₃H, —SO—NH₂, —SO₂—NH₂, —CO—NH—OH, —CO—NH—CN, —CO—NH—SO—(C₁₋₄ alkyl), —CO—NH—SO₂—(C₁₋₄ alkyl), —CO—NH—SO—(C₃₋₇ cycloalkyl), —CO—NH—SO₂—(C₃₋₇ cycloalkyl), —CO—NH—SO-aryl, —CO—NH—SO₂-aryl, —CO—NH—SO-heteroaryl, —CO—NH—SO₂-heteroaryl, —CO—NH—SO—(C₁₋₄ alkylene)aryl, —CO—NH—SO₂—(C₁₋₄ alkylene)aryl, —CO—NH—SO—(C₁₋₄ alkylene)heteroaryl, or —CO—NH—SO₂—(C₁₋₄ alkylene)heteroaryl, wherein the aryl moiety comprised in said —CO—NH—SO-aryl, said —CO—NH—SO₂-aryl, said —CO—NH—SO—(C₁₋₄ alkylene)aryl, or said —CO—NH—SO₂—(C₁₋₄ alkylene)aryl is optionally substituted with one or more groups independently selected from C₁₋₄ alkyl, halogen, —CF₃, —CN, —NO₂, —N₃, —OH, —O(C₁₋₄ alkyl), —SH, —S(C₁₋₄ alkyl), —NH₂, —NH(C₁₋₄ alkyl), or —N(C₁₋₄ alkyl)(C₁₋₄ alkyl), and further wherein the heteroaryl moiety comprised in said —CO—NH—SO-heteroaryl, said —CO—NH—SO₂-heteroaryl, said —CO—NH—SO—(C₁₋₄ alkylene)heteroaryl, or said —CO—NH—SO₂—(C₁₋₄ alkylene)heteroaryl is optionally substituted with one or more groups independently selected from C₁₋₄ alkyl, halogen, —CF₃, —CN, —NO₂, —N₃, —OH, —O(C₁₋₄ alkyl), —SH, —S(C₁₋₄ alkyl), —NH₂, —NH(C₁₋₄ alkyl), or —N(C₁₋₄ alkyl)(C₁₋₄ alkyl);
L¹¹ is C₁₋₆ alkylene or C₂₋₆ alkenylene, wherein said alkylene or said alkenylene is optionally substituted with one or more groups independently selected from halogen, —CF₃, —CN, —OH, —O(C₁₋₄ alkyl), —NH₂, —NH(C₁₋₄ alkyl), or —N(C₁₋₄ alkyl)(C₁₋₄ alkyl), and further wherein one —CH₂— unit comprised in said alkylene or said alkenylene is optionally replaced by C₃₋₇ cycloalkylene;
L¹² is C₁₋₆ alkylene or C₂₋₆ alkenylene, wherein said alkylene or said alkenylene is optionally substituted with one or more groups independently selected from halogen, —CF₃, —CN, —OH, —O(C₁₋₄ alkyl), —NH₂, —NH(C₁₋₄ alkyl), or —N(C₁₋₄ alkyl)(C₁₋₄ alkyl);
L¹³ is C₁₋₆ alkylene or C₂₋₆ alkenylene, wherein said alkylene or said alkenylene is optionally substituted with one or more groups independently selected from halogen, —CF₃, —CN, —OH, —O(C₁₋₄ alkyl), —NH₂, —NH(C₁₋₄ alkyl), or —N(C₁₋₄ alkyl)(C₁₋₄ alkyl);
n is an integer of 0 to 4; and
m is an integer of 0 to 4;
or a pharmaceutically acceptable salt or solvate thereof.

23. The pharmaceutical composition of claim 22 or the method of claim 22 or the leukotriene antagonist or the leukotriene receptor antagonist for use according to claim 22, wherein each R¹¹ is independently selected from halogen, —CF₃, or —CN.

24. The pharmaceutical composition of claim 22 or 23 or the method of claim 22 or 23 or the leukotriene antagonist or the leukotriene receptor antagonist for use according to claim 22 or 23, wherein R¹³ is selected from —COOH, tetrazolyl, —SO₃H, —SO—NH₂, —SO₂—NH₂, —CO—NH—OH, or —CO—NH—CN.

25. The pharmaceutical composition of any of claims 22 to 24 or the method of any of claims 22 to 24 or the leukotriene antagonist or the leukotriene receptor antagonist for use according to any of claims 22 to 24, wherein R¹³ is —COOH.

26. The pharmaceutical composition of any of claims 22 to 25 or the method of any of claims 22 to 25 or the leukotriene antagonist or the leukotriene receptor antagonist for use according to any of claims 22 to 25, wherein L¹¹ is C₁₋₄ alkylene, wherein one —CH₂— unit comprised in said C₁₋₄ alkylene is replaced by 1,1-cyclopropylene.

27. The pharmaceutical composition of any of claims 22 to 26 or the method of any of claims 22 to 26 or the leukotriene antagonist or the leukotriene receptor antagonist for use according to any of claims 22 to 26, wherein L¹² is —CH₂CH₂— or —CH₂CH₂CH₂—.

28. The pharmaceutical composition of any of claims 22 to 27 or the method of any of claims 22 to 27 or the leukotriene antagonist or the leukotriene receptor antagonist for use according to any of claims 22 to 27, wherein L¹³ is C₁₋₄ alkylene.

29. The pharmaceutical composition of any of claims 22 to 28 or the method of any of claims 22 to 28 or the leukotriene antagonist or the leukotriene receptor antagonist for use according to any of claims 22 to 28, wherein n is 1.

30. The pharmaceutical composition of any of claims 22 to 29 or the method of any of claims 22 to 29 or the leukotriene antagonist or the leukotriene receptor antagonist for use according to of any of claims 22 to 29, wherein m is 0.

31. The pharmaceutical composition of claim 22 or the method of claim 22 or the leukotriene antagonist or the leukotriene receptor antagonist for use according to claim 22, wherein the leukotriene antagonist or leukotriene receptor antagonist is a compound having the following formula: 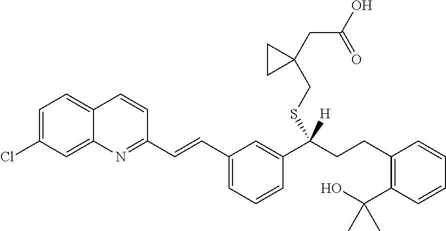 [SMILES: CC(C)(O)c1ccccc1CC[C@@H](SCC1(CC(=O)O)CC1)c1cccc(/C=C/c2ccc3ccc(Cl)cc3n2)c1]
or a pharmaceutically acceptable salt or solvate thereof.

32. The pharmaceutical composition of any of claims 1 to 7 or the method of any of claims 8 to 14 or the leukotriene antagonist or the leukotriene receptor antagonist for use according to any of claims 15 to 21, wherein the leukotriene antagonist or leukotriene receptor antagonist is a compound of the following formula (II): 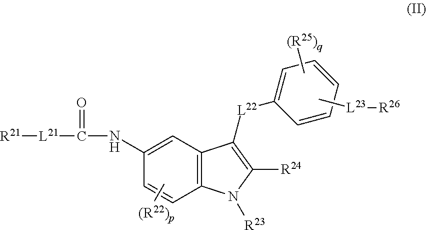
wherein:
R²¹ is selected from C₁₋₆ alkyl, C₂₋₆ alkenyl, C₂₋₆ alkynyl, C₃₋₇ cycloalkyl, —(C₁₋₄ alkylene)-(C₃₋₇ cycloalkyl), phenyl, or —(C₁₋₄ alkylene)-phenyl;
each R²² is independently selected from C₁₋₄ alkyl, halogen, —CF₃, —CN, —NO₂, —N₃, —OH, —O(C₁₋₄ alkyl), —SH, —S(C₁₋₄ alkyl), —NH₂, —NH(C₁₋₄ alkyl), or —N(C₁₋₄ alkyl)(C₁₋₄ alkyl);
R²³ is selected from hydrogen, C₁₋₆ alkyl, C₂₋₆ alkenyl, C₂₋₆ alkynyl, —(C₁₋₄ alkylene)-O—(C₁₋₄ alkyl), —(C₁₋₄ alkylene)-COOH, —(C₁₋₄ alkylene)-CONH₂, —(C₁₋₄ alkylene)-CONH—(C₁₋₄ alkyl), —(C₁₋₄ alkylene)-CON(C₁₋₄ alkyl)(C₁₋₄ alkyl), C₃₋₇ cycloalkyl, —(C₁₋₄ alkylene)-(C₃₋₇ cycloalkyl), —CO—(C₁₋₄ alkyl), or —(C₁₋₄ alkylene)-phenyl;
R²⁴ is selected from hydrogen, C₁₋₄ alkyl, halogen, —CF₃, —CN, —NO₂, —N₃, —OH, —O(C₁₋₄ alkyl), —SH, —S(C₁₋₄ alkyl), —NH₂, —NH(C₁₋₄ alkyl), or —N(C₁₋₄ alkyl)(C₁₋₄ alkyl);
each R²⁵ is independently selected from C₁₋₄ alkyl, halogen, —CF₃, —CN, —NO₂, —N₃, —OH, —O(C₁₋₄ alkyl), —SH, —S(C₁₋₄ alkyl), —NH₂, —NH(C₁₋₄ alkyl), or —N(C₁₋₄ alkyl)(C₁₋₄ alkyl);
R²⁶ is selected from —COOH, tetrazolyl, —SO₃H, —SO—NH₂, —SO₂—NH₂, —CO—NH—OH, —CO—NH—CN, —CO—NH—SO—(C₁₋₄ alkyl), —CO—NH—SO₂—(C₁₋₄ alkyl), —CO—NH—SO—(C₃₋₇ cycloalkyl), —CO—NH—SO₂—(C₃₋₇ cycloalkyl), —CO—NH—SO-aryl, —CO—NH—SO₂-aryl, —CO—NH—SO-heteroaryl, —CO—NH—SO₂-heteroaryl, —CO—NH—SO—(C₁₋₄ alkylene)aryl, —CO—NH—SO₂—(C₁₋₄ alkylene)aryl, —CO—NH—SO—(C₁₋₄ alkylene)heteroaryl, or —CO—NH—SO₂—(C₁₋₄ alkylene)heteroaryl, wherein the aryl moiety comprised in said —CO—NH—SO-aryl, said —CO—NH—SO₂-aryl, said —CO—NH—SO—(C₁₋₄ alkylene)aryl, or said —CO—NH—SO₂—(C₁₋₄ alkylene)aryl is optionally substituted with one or more groups independently selected from C₁₋₄ alkyl, halogen, —CF₃, —CN, —NO₂, —N₃, —OH, —O(C₁₋₄ alkyl), —SH, —S(C₁₋₄ alkyl), —NH₂, —NH(C₁₋₄ alkyl), or —N(C₁₋₄ alkyl)(C₁₋₄ alkyl), and further wherein the heteroaryl moiety comprised in said —CO—NH—SO-heteroaryl, said —CO—NH—SO₂-heteroaryl, said —CO—NH—SO—(C₁₋₄ alkylene)heteroaryl, or said —CO—NH—SO₂—(C₁₋₄ alkylene)heteroaryl is optionally substituted with one or more groups independently selected from C₁₋₄ alkyl, halogen, —CF₃, —CN, —NO₂, —N₃, —OH, —O(C₁₋₄ alkyl), —SH, —S(C₁₋₄ alkyl), —NH₂, —NH(C₁₋₄ alkyl), or —N(C₁₋₄ alkyl)(C₁₋₄ alkyl);
L²¹ is a covalent bond, —O—, —S—, —NH—, or —N(C₁₋₄ alkyl)-;
L²² is a covalent bond or C₁₋₄ alkylene;
L²³ is a covalent bond or C₁₋₄ alkylene;
p is an integer of 0 to 3; and
q is an integer of 0 to 4;
or a pharmaceutically acceptable salt or solvate thereof.

33. The pharmaceutical composition of claim 32 or the method of claim 32 or the leukotriene antagonist or the leukotriene receptor antagonist for use according to claim 32, wherein R²¹ is C₃₋₇ cycloalkyl.

34. The pharmaceutical composition of claim 32 or 33 or the method of claim 32 or 33 or the leukotriene antagonist or the leukotriene receptor antagonist for use according to claim 32 or 33, wherein R²³ is C₁₋₄ alkyl.

35. The pharmaceutical composition of any of claims 32 to 34 or the method of any of claims 32 to 34 or the leukotriene antagonist or the leukotriene receptor antagonist for use according to any of claims 32 to 34, wherein R²⁴ is hydrogen.

36. The pharmaceutical composition of any of claims 32 to 35 or the method of any of claims 32 to 35 or the leukotriene antagonist or the leukotriene receptor antagonist for use according to any of claims 32 to 35, wherein R²⁶ is selected from —COOH, —CO—NH—SO₂-aryl, —CO—NH—SO₂-heteroaryl, —CO—NH—SO₂—(C₁₋₄ alkylene)aryl, or —CO—NH—SO₂—(C₁₋₄ alkylene)heteroaryl, wherein the aryl moiety comprised in said —CO—NH—SO₂-aryl or said —CO—NH—SO₂—(C₁₋₄ alkylene)aryl is optionally substituted with one or more groups independently selected from C₁₋₄ alkyl, halogen, —CF₃, —CN, —NO₂, —N₃, —OH, —O(C₁₋₄ alkyl), —SH, —S(C₁₋₄ alkyl), —NH₂, —NH(C₁₋₄ alkyl), or —N(C₁₋₄ alkyl)(C₁₋₄ alkyl), and further wherein the heteroaryl moiety comprised in said —CO—NH—SO₂-heteroaryl or said —CO—NH—SO₂—(C₁₋₄ alkylene)heteroaryl is optionally substituted with one or more groups independently selected from C₁₋₄ alkyl, halogen, —CF₃, —CN, —NO₂, —N₃, —OH, —O(C₁₋₄ alkyl), —SH, —S(C₁₋₄ alkyl), —NH₂, —NH(C₁₋₄ alkyl), or —N(C₁₋₄ alkyl)(C₁₋₄ alkyl).

37. The pharmaceutical composition of any of claims 32 to 36 or the method of any of claims 32 to 36 or the leukotriene antagonist or the leukotriene receptor antagonist for use according to any of claims 32 to 36, wherein R²⁶ is —CO—NH—SO₂-phenyl, wherein the phenyl moiety comprised in said —CO—NH—SO₂-phenyl is optionally substituted with one or two groups independently selected from C₁₋₄ alkyl, halogen, —CF₃, —CN, —NO₂, —N₃, —OH, —O(C₁₋₄ alkyl), —SH, —S(C₁₋₄ alkyl), —NH₂, —NH(C₁₋₄ alkyl), or —N(C₁₋₄ alkyl)(C₁₋₄ alkyl).

38. The pharmaceutical composition of any of claims 32 to 37 or the method of any of claims 32 to 37 or the leukotriene antagonist or the leukotriene receptor antagonist for use according to any of claims 32 to 37, wherein L²¹ is —O—.

39. The pharmaceutical composition of any of claims 32 to 38 or the method of any of claims 32 to 38 or the leukotriene antagonist or the leukotriene receptor antagonist for use according to any of claims 32 to 38, wherein L²² is methylene.

40. The pharmaceutical composition of any of claims 32 to 39 or the method of any of claims 32 to 39 or the leukotriene antagonist or the leukotriene receptor antagonist for use according to any of claims 32 to 39, wherein L²³ is a covalent bond.

41. The pharmaceutical composition of any of claims 32 to 40 or the method of any of claims 32 to 40 or the leukotriene antagonist or the leukotriene receptor antagonist for use according to any of claims 32 to 40, wherein the moiety -L²³-R²⁶ is bound to the phenyl moiety comprised in the compound of formula (II) in para position with respect to the group L²².

42. The pharmaceutical composition of any of claims 32 to 41 or the method of any of claims 32 to 41 or the leukotriene antagonist or the leukotriene receptor antagonist for use according to any of claims 32 to 41, wherein p is 0.

43. The pharmaceutical composition of any of claims 32 to 42 or the method of any of claims 32 to 42 or the leukotriene antagonist or the leukotriene receptor antagonist for use according to any of claims 32 to 42, wherein q is 0.

44. The pharmaceutical composition of claim 32 or the method of claim 32 or the leukotriene antagonist or the leukotriene receptor antagonist for use according to claim 32, wherein the leukotriene antagonist or leukotriene receptor antagonist is a compound having the following formula: 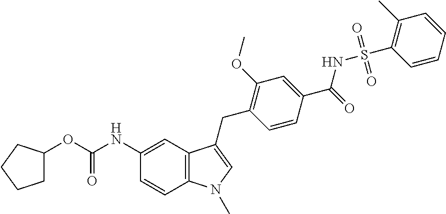 [SMILES: COc1cc(C(=O)NS(=O)(=O)c2ccccc2C)ccc1Cc1cn(C)c2ccc(NC(=O)OC3CCCC3)cc12]
or a pharmaceutically acceptable salt or solvate thereof.

45. The pharmaceutical composition of any of claims 1 to 7 or the method of any of claims 8 to 14 or the leukotriene antagonist or the leukotriene receptor antagonist for use according to any of claims 15 to 21, wherein the leukotriene antagonist or leukotriene receptor antagonist is a compound of the following formula (III): 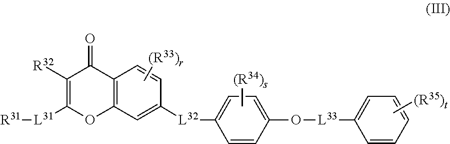
wherein:
R³¹ is selected from —COOH, tetrazolyl, —SO₃H, —SO—NH₂, —SO₂—NH₂, —CO—NH—OH, —CO—NH—CN, —CO—NH—SO—(C₁₋₄ alkyl), —CO—NH—SO₂—(C₁₋₄ alkyl), —CO—NH—SO—(C₃₋₇ cycloalkyl), —CO—NH—SO₂—(C₃₋₇ cycloalkyl), —CO—NH—SO-aryl, —CO—NH—SO₂-aryl, —CO—NH—SO-heteroaryl, —CO—NH—SO₂-heteroaryl, —CO—NH—SO—(C₁₋₄ alkylene)aryl, —CO—NH—SO₂—(C₁₋₄ alkylene)aryl, —CO—NH—SO—(C₁₋₄ alkylene)heteroaryl, or —CO—NH—SO₂—(C₁₋₄ alkylene)heteroaryl, wherein the aryl moiety comprised in said —CO—NH—SO-aryl, said —CO—NH—SO₂-aryl, said —CO—NH—SO—(C₁₋₄ alkylene)aryl, or said —CO—NH—SO₂—(C₁₋₄ alkylene)aryl is optionally substituted with one or more groups independently selected from C₁₋₄ alkyl, halogen, —CF₃, —CN, —NO₂, —N₃, —OH, —O(C₁₋₄ alkyl), —SH, —S(C₁₋₄ alkyl), —NH₂, —NH(C₁₋₄ alkyl), or —N(C₁₋₄ alkyl)(C₁₋₄ alkyl), and further wherein the heteroaryl moiety comprised in said —CO—NH—SO-heteroaryl, said —CO—NH—SO₂-heteroaryl, said —CO—NH—SO—(C₁₋₄ alkylene)heteroaryl, or said —CO—NH—SO₂—(C₁₋₄ alkylene)heteroaryl is optionally substituted with one or more groups independently selected from C₁₋₄ alkyl, halogen, —CF₃, —CN, —NO₂, —N₃, —OH, —O(C₁₋₄ alkyl), —SH, —S(C₁₋₄ alkyl), —NH₂, —NH(C₁₋₄ alkyl), or —N(C₁₋₄ alkyl)(C₁₋₄ alkyl);
R³² is selected from hydrogen, C₁₋₄ alkyl, halogen, —CF₃, —CN, —NO₂, —N₃, —OH, —O(C₁₋₄ alkyl), —SH, —S(C₁₋₄ alkyl), —NH₂, —NH(C₁₋₄ alkyl), or —N(C₁₋₄ alkyl)(C₁₋₄ alkyl);
each R³³ is independently selected from C₁₋₄ alkyl, halogen, —CF₃, —CN, —NO₂, —N₃, —OH, —O(C₁₋₄ alkyl), —SH, —S(C₁₋₄ alkyl), —NH₂, —NH(C₁₋₄ alkyl), or —N(C₁₋₄ alkyl)(C₁₋₄ alkyl);
each R³⁴ is independently selected from C₁₋₄ alkyl, halogen, —CF₃, —CN, —NO₂, —N₃, —OH, —O(C₁₋₄ alkyl), —SH, —S(C₁₋₄ alkyl), —NH₂, —NH(C₁₋₄ alkyl), or —N(C₁₋₄ alkyl)(C₁₋₄ alkyl);
each R³⁵ is independently selected from C₁₋₄ alkyl, halogen, —CF₃, —CN, —NO₂, —N₃, —OH, —O(C₁₋₄ alkyl), —SH, —S(C₁₋₄ alkyl), —NH₂, —NH(C₁₋₄ alkyl), or —N(C₁₋₄ alkyl)(C₁₋₄ alkyl);
L³¹ is a covalent bond or C₁₋₄ alkylene;
L³² is C₁₋₄ alkylene, C₂₋₄ alkenylene, —O—, —S—, —NH—, —N(C₁₋₄ alkyl)-, —CO—, —CO—NH—, —NH—CO—, —CO—N(C₁₋₄ alkyl)-, or —N(C₁₋₄ alkyl)-CO—, wherein one or two —CH₂— units comprised in said C₁₋₄ alkylene or said C₂₋₄ alkenylene are each optionally replaced by a group selected independently from —O—, —S—, —NH—, —N(C₁₋₄ alkyl)-, or —CO—;
L³³ is C₁₋₁₀ alkylene;
r is an integer of 0 to 3;
s is an integer of 0 to 4; and
t is an integer of 0 to 5;
or a pharmaceutically acceptable salt or solvate thereof.

46. The pharmaceutical composition of claim 45 or the method of claim 45 or the leukotriene antagonist or the leukotriene receptor antagonist for use according to claim 45, wherein R³¹ is selected from —COOH, tetrazolyl, —SO₃H, —SO—NH₂, —SO₂—NH₂, —CO—NH—OH, or —CO—NH—CN.

47. The pharmaceutical composition of claim 45 or 46 or the method of claim 45 or 46 or the leukotriene antagonist or the leukotriene receptor antagonist for use according to claim 45 or 46, wherein R³¹ is tetrazolyl.

48. The pharmaceutical composition of any of claims 45 to 47 or the method of any of claims 45 to 47 or the leukotriene antagonist or the leukotriene receptor antagonist for use according to any of claims 45 to 47, wherein R³² is hydrogen.

49. The pharmaceutical composition of any of claims 45 to 48 or the method of any of claims 45 to 48 or the leukotriene antagonist or the leukotriene receptor antagonist for use according to any of claims 45 to 48, wherein L³¹ is a covalent bond.

50. The pharmaceutical composition of any of claims 45 to 49 or the method of any of claims 45 to 49 or the leukotriene antagonist or the leukotriene receptor antagonist for use according to any of claims 45 to 49, wherein L³² is —CO—NH—, —NH—CO—, —CO—N(C₁₋₄ alkyl)-, or —N(C₁₋₄ alkyl)-CO—.

51. The pharmaceutical composition of any of claims 45 to 50 or the method of any of claims 45 to 50 or the leukotriene antagonist or the leukotriene receptor antagonist for use according to any of claims 45 to 50, wherein L³³ is —(CH₂)₃₋₆—.

52. The pharmaceutical composition of any of claims 45 to 51 or the method of any of claims 45 to 51 or the leukotriene antagonist or the leukotriene receptor antagonist for use according to any of claims 45 to 51, wherein r is 0.

53. The pharmaceutical composition of any of claims 45 to 52 or the method of any of claims 45 to 52 or the leukotriene antagonist or the leukotriene receptor antagonist for use according to any of claims 45 to 52, wherein s is 0.

54. The pharmaceutical composition of any of claims 45 to 53 or the method of any of claims 45 to 53 or the leukotriene antagonist or the leukotriene receptor antagonist for use according to any of claims 45 to 53, wherein t is 0.

55. The pharmaceutical composition of claim 45 or the method of claim 45 or the leukotriene antagonist or the leukotriene receptor antagonist for use according to claim 45, wherein the leukotriene antagonist or leukotriene receptor antagonist is a compound having the following formula: 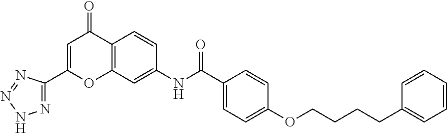 [SMILES: O=C(Nc1ccc2c(=O)cc(-c3nn[nH]n3)oc2c1)c1ccc(OCCCCc2ccccc2)cc1]
or a pharmaceutically acceptable salt or solvate thereof.
